(19) United States
(12) Patent Application Publication
(10) Pub. No.: US 2026/0327879 A1
(43) Pub. Date: Oct. 22, 2026

(54) LAUNDRY TREATING APPARATUS AND CONTROL METHOD THEREFOR

(71) Applicant: LG ELECTRONICS INC., Seoul (KR)

(72) Inventors: Dongkil CHOI, Seoul (KR); Injae HAN, Seoul (KR); Kanghun LEE, Seoul (KR); Yeongeun KIM, Seoul (KR); Woore KIM, Seoul (KR)

(73) Assignee: LG ELECTRONICS INC., Seoul (KR)

(21) Appl. No.: 19/112,174

(22) PCT Filed: Dec. 15, 2023
(86) PCT No.: PCT/KR2023/020760

§ 371 Date: Mar. 14, 2025

(30) Foreign Application Priority Data
Dec. 15, 2022 (KR) 10-2022-0175848

(51) Int. Cl. D06F 21/02; A61L 2/04; A61L 103/50; D06F 33/32; D06F 39/04; D06F 101/12; D06F 103/18; D06F 105/02; D06F 105/28; D06F 105/48; D06F 105/56; H05B 6/10

(52) U.S. Cl. CPC D06F 21/02; A61L 2/04; D06F 33/32; D06F 39/04; H05B 6/10; A61L 2103/50; D06F 2101/12; D06F 2103/18; D06F 2105/02; D06F 2105/28; D06F 2105/48; D06F 2105/56

(57) ABSTRACT

Abstract: To solve the above-described problems, the present invention provides a laundry treating apparatus in which, during a sterilization course, the drum is driven to operate an induction module, and thus, laundry can be washed simultaneously with performing the sterilization course.

### TECHNICAL FIELD

The present disclosure relates to a laundry treating apparatus and a control method therefor.

### BACKGROUND ART

In general, a laundry treating apparatus is capable of performing a wash cycle to remove dirt on clothes using water and detergent.

However, bacteria or mites on the clothes may not be eliminated with only water and detergent, which may cause an allergic reaction to the user. Even stains on the clothes may not be removed with only water and detergent.

To address this issue, conventional laundry treating apparatuses may heat the clothes with the clothes submerged in water, or perform a heating cycle or boiling cycle in which the clothes are placed in high-temperature water supplied for a period of time.

Submerging the clothes in high-temperature water not only increases the solubility of the detergent, but also increases the cleaning power of the detergent to separate dirt. Furthermore, when the clothes are immersed in high-temperature water for longer than the sanitization time, more than 99 percent of the bacteria and mites present on the clothes may be eradicated.

Of course, one may consider increasing the temperature of the clothes by supplying steam or the like to the clothes. However, steam holds less heat capacity than water even at high temperatures because it has a smaller mass.

Therefore, in order to increase the cleaning power of the detergent or to eradicate bacteria and mites, the heating cycle in which clothes are immersed in high-temperature water may be more effective than the steam cycle in which steam is supplied to the clothes. The temperature of the water performing the heating cycle may be set to 50 °C (hereinafter, the sanitization temperature) or higher.

FIG. 1 illustrates a conventional laundry treating apparatus performing the heating cycle according to an embodiment.

Referring to FIG. 1-(a), clothing is accommodated in a drum 30, and a heater H configured to heat water may be arranged in a tub 20 accommodating the drum 30.

Referring to FIG. 1-(b), in order to perform the heating cycle, the conventional laundry treating apparatus may supply water through a water supply part 50 up to a water level at which the clothes are completely submerged in the water.

The water supply part 50 may supply high-temperature water from the outset. However, the conventional laundry treating apparatus may supply cold water and then heat the heater H to heat the water to the sanitization temperature or higher.

Referring to FIG. 1-(c), when the water accommodated in the tub 20 is heated to the sterilization temperature or higher, the water does not only supply high-temperature heat to the clothes, but also dissipates heat to the outside of the tub 20.

Part of the heat dissipated to the outside of the tub 20 is transferred to a drive part 40, which rotates the drum 30.

When the drive part 40 rotates the drum 30 at a water level at which the clothes are submerged in water, a substantial load is placed on the drive part 40, causing the temperature of the drive part 40 to rise. In this case, if the heat generated from the high-temperature water is transferred to the drive part 40, the temperature of the drive part 40 will rise more quickly even when the drive part 40 is driven only a little.

As a result, the drive part 40 may be difficult to drive at normal power during the heating cycle or may have durability issues.

Therefore, it is common for a conventional laundry treating apparatus to be controlled to minimize the control of the drive part 40 when heating the water at a water level at which clothing may be submerged in the water.

FIG. 2 illustrates an actual operation rate of the drive part in a heating cycle of a conventional laundry treating apparatus.

When water reaches a level at which the clothing can be submerged after start of the heating cycle, and the water is heated by the heater H or the like, the spinning of the drum 30 may be minimized.

Of course, the drum 30 may be kept completely stationary. However, in order to prevent specific parts of the clothes from being excessively heated, the drive part 40 may be rotated intermittently.

In the heating cycle, the conventional laundry treating apparatus drives the drive part 40 for a drive time ta and then stops driving the drive part 40 for a stop time tb. In this case, the stop time may be set much longer than the drive time such that the actual operation rate of the drive part 40 is set to be lower than 50% in the heating cycle.

For example, the stop time tb may be set to be more than twice as long as the drive time ta.

FIG. 3 illustrates a control method by which a conventional laundry treating apparatus performs any course in which the heating cycle is performed.

The conventional laundry treating apparatus cannot spin the drum 30 in the heating cycle, and thus cannot deliver sufficient physical force to the clothes. Therefore, the conventional laundry treating apparatus has a fundamental limitation that it cannot perform washing and rinsing of the clothes simultaneously in the heating cycle.

Therefore, when the conventional laundry treating apparatus performs any course in which a wash cycle (C2), a rinse cycle (C3), and a dehydration cycle (C4) are performed, and the course corresponds to a boiling course or an anti-allergy course including a heating cycle (C1), the conventional laundry treating apparatus should perform the heating cycle independently.

As a result, when the conventional laundry treating apparatus performs any course including the heating cycle, not only does the wash delay inevitably occur, but also the water used for the heating cycle cannot be utilized for the wash cycle (C2), rinse cycle (C4), and the like. Thus, energy waste becomes severe.

In this regard, a laundry treating apparatus capable of quickly heating the drum with an induction module I has been introduced (see Korean Patent Application Publication No. 10-2020-0042821 and Korean Patent Application Publication No. 10-2020-0073060).

This conventional laundry treating apparatus has the advantage of saving energy by quickly raising the temperature of the drum compared to the method of heating water with the heater H.

However, the conventional laundry treating apparatus merley heats the outer circumferential surface of the drum 30 facing the induction module I. Therefore, the conventional laundry treating apparatus has a fundamental limitation that the drum 30 must be spun when driving the induction module I in order to transfer the heat of the heated drum 30 to the clothing.

Therefore, the conventional laundry treating apparatus applies a control method for driving the induction module I at a water level at which no water is introduced into the drum 30. No embodiment of driving the induction module I while the clothes are completely submerged in water is known so far.

When the heating cycle of the conventional laundry treating apparatus that has a low actual operation rate is applied to a conventional laundry treating apparatus having a induction module I, the actual operation rate of the induction module I is also lowered, resulting in a further delay in the heating cycle. Thus, this cycle is difficult to apply to real products.

Furthermore, in the case where the wash cycle and the rinse cycle are performed simultaneously while the induction module I is driven, an excessive load may occur on the drive part 40. Thus, the conventional laundry treating apparatus does not consider an embodiment in which the induction module I is driven while the wash cycle and rinse cycle are performed with the clothes submerged in water.

In the conventional laundry treating apparatus configured in any way, spinning the drum at high speed along with introduced detergent in the wash cycle may generate a lot of bubbles although it may increase the washing performance.

Therefore, the conventional laundry treating apparatus cannot increase the spin speed of the drum beyond a certain level during the wash cycle.

### DISCLOSURE

### Technical Problem

An object of the present disclosure is to provide a control method for a laundry treating apparatus capable of preventing excessive load on a drive part that spins a drum even when water is heated by an induction module with clothes submerged in water.

Another object of the present disclosure is to provide a control method for a laundry treating apparatus capable of preventing bubbles from forming or removing formed bubbles even when the drum is spun with detergent added to water.

Another object of the present disclosure is to provide a control method for a laundry treating apparatus capable of performing at least one of a wash cycle and a rinse cycle simultaneously in a heating cycle.

### Technical Solution

The objects of the present disclosure can be achieved by providing a laundry treating apparatus that drives a drum for IH operation when performing a sanitization course. Thus, the laundry treating apparatus according to the present disclosure may wash clothing while performing the sanitization course.

The laundry treating apparatus according to the present disclosure may have a period in which it generates an acceleration force of 1G or more to reduce the load on the drive part. When the acceleration force of 1 G or more is applied inside the drum, the load on the drive part may be reduced due to the absence of falling and repositioning of clothing.

The laundry treating apparatus according to the present disclosure may control the driving method of the drum differently depending on the water temperature.

The laundry treating apparatus according to the present disclosure may wash clothing while heating the drum, prevent bubbles from being generated inside the drum, or eliminate the bubbles.

The laundry treating apparatus according to the present disclosure may spin the drum to generate an acceleration force of 1 G or more at a bubble removal temperature (e.g., 60 °C) or higher, and may spin the drum to generate an acceleration force of 1 G or less at the bubble removal temperature (e.g., 60 °C) or lower.

The laundry treating apparatus according to the present disclosure may spin the drum to generate an acceleration force of 1G or more at a drive part load temperature (e.g., 45 °C) or higher. In this case, the speed of 1G or more and the actual operation rate may be set to be large to reduce load on the drive part.

The laundry treating apparatus according to the present disclosure may spin the drum to generate an acceleration force of 1G or less at a drive part load temperature (e.g., 45 °C) or lower.

The laundry treating apparatus according to the present disclosure may vary the speed of the drum and the actual operation rate of the drive part in the speed range of 1G or less to enhance the washing effect.

The laundry treating apparatus according to the present disclosure may control the drive part at the same speed and actual operation rate as the existing wash or rinse cycle when the water heating cycle is terminated. In other words, it may not unnecessarily drive the drum to generate an acceleration force of more than 1G in the wash cycle and rinse cycle.

The present disclosure also provides a control method for a laundry treating apparatus capable of performing at least one of a wash cycle and a rinse cycle while heating a drum with an induction module.

The object of the present disclosure can be achieved by providing a laundry treating apparatus including a cabinet having an opening in a front thereof, a tub arranged inside the cabinet to store water, a water supply valve coupled to the cabinet to supply the water, a drum rotatably disposed inside the tub to accommodate clothing, the drum including a conductor, a drive part coupled to the tub to spin the drum, an induction module coupled to an outer circumferential surface of the tub to generate a magnetic field to heat the drum, a controller configured to control at least one of the water supply valve, the drive part, or the induction module to perform a heating cycle of heating an inside of the drum to a sanitization temperature or higher at a water level submerging the clothing in water.

In the heating cycle, the controller may arrange at least a portion of a period of spinning the drum at a first spin speed or higher causing the clothing to be stuck to an inner wall of the drum while rotating one or more revolutions.

In the heating cycle, the controller may be configured to stop driving the induction module when the drum stops.

In the heating cycle, the controller may repeat stopping the drum after spinning the drum at the first spin speed or higher.

The laundry treating apparatus may further include a water level sensor configured to sense a water level in the tub and communicate the same to the controller, wherein the controller may sense the water level in the tub in a period of stopping the drum.

In the heating cycle, the controller may further arrange a period of spinning the drum at a speed lower than the first spin speed such that the clothing is separated from the inner wall of the drum.

Based on a temperature inside the tub reaching the sanitization temperature or load temperature or higher in the heating cycle, the controller may arrange the period of spinning the drum at the first spin speed or higher to be longer than the period of spinning the drum at the speed lower than the first spin speed.

Based on a temperature inside the tub reaching the sanitization temperature or the load temperature or higher in the heating cycle, the controller may set a time for continuous spinning of the drum at the first spin speed or higher to be longer than a time for continuous spinning of the drum at a speed lower than the first spin speed.

Based on a temperature inside the tub reaching the sanitization temperature or the load temperature or higher in the heating cycle, the controller may arrange the period of spinning the drum at the speed lower than the first spin speed in the period of spinning the drum at the first spin speed or higher.

Based on a temperature inside the tub reaching the sanitization temperature or the load temperature or higher in the heating cycle, the controller may set the number of times of repeating stopping the drum after spinning the drum at the first spin speed or higher in the heating cycle to be greater than the number of times of repeating stopping the drum after spinning the drum at the speed lower than the first spin speed.

Based on a temperature inside the tub being lower than a load temperature lower than the sanitization temperature in the heating cycle, the controller may arrange the period of spinning the drum at the first spin speed or higher to be shorter than the period of spinning the drum at the speed lower than the first spin speed.

Based on a temperature inside the tub being lower than a load temperature lower than the sanitization temperature in the heating cycle, the controller may set a time for continuous spinning of the drum at a speed lower than the first spin speed to be equal to or longer than a time for continuous spinning of the drum at the first spin speed or higher.

Based on a temperature inside the tub being lower than a load temperature lower than the sanitization temperature in the heating cycle, the controller may set the number of times of repeating stopping the drum after spinning the drum at the first spin speed or higher to be less than the number of times of repeating stopping the drum after spinning the drum at the speed lower than the first spin speed.

Based on the drum being spun at the speed lower than the first spin speed, the controller may arrange at least a portion of a period of spinning the drum at a second spin speed lower than the first spin speed, and a period of spinning the drum at a third spin speed lower than the second spin speed.

In spinning the drum at the speed lower than the first spin speed, the controller may arrange at least a portion of a period of spinning the drum at the second spin speed and stopping the drum, and a period of spinning the drum at the third spin speed and stopping the drum.

In spinning the drum at the speed lower than the first spin speed, the controller may arrange a combination of the period of spinning the drum at the second spin speed, and the period of spinning the drum at the third spin speed.

In spinning the drum at the speed lower than the first spin speed, the controller may further arrange, in combination, a period of spinning the drum at a fourth spin speed lower than the third spin speed.

The load temperature may be set to a temperature at which bubbles generated in the drum is removed.

The load temperature may be set based on a temperature at which the drive part enters an overloaded state due to heat transferred from the tub during operation of the drive part.

In the heating cycle, the controller may set a time for spinning of the drum with the clothing to be longer than a time for stopping of the drum.

The controller performs a control operation to drive the induction module when the drum spins, and block driving of the induction module when the drum stops.

When a temperature inside the tub reaches the sanitization temperature or a temperature higher than or equal to the load temperature lower than the sanitization temperature in the heating cycle, the controller may set a larger ratio of the time for spinning of the drum to the time for stopping of the drum than when the temperature inside the tub is lower than the load temperature.

The controller may arrange a period of spinning the drum in the heating cycle at a first spin speed or higher and stopping the drum, the first spin speed causing the clothing to be stuck to an inner wall of the drum while rotating at one or more revolutions, and set a time for spinning of the drum at the first spin speed or higher to be longer than a time for stopping of the drum.

When a temperature inside the tub reaches the sanitization temperature or a temperature higher than or equal to the load temperature lower than the sanitization temperature in the heating cycle, the controller may stop the drum after spinning the drum at the first spin speed more than when the temperature inside the tub is lower than the load temperature.

The controller may further arrange a period of spinning the drum at a speed lower than the first spin speed and stopping the drum in the heating cycle, and set a time for spinning of the drum at the speed lower than the first spin speed to be longer than a time for stopping of the drum.

When a temperature inside the tub reaches the sanitization temperature or a temperature higher than or equal to the load temperature lower than the sanitization temperature in the heating cycle, the controller may set a smaller ratio of the time for spinning of the drum to the time for stopping of the drum at the speed lower than the first spin speed than when the temperature inside the tub is lower than the load temperature.

In a cycle after the heating cycle, the controller may set the time for stopping of the drum to be longer than the time for spinning of the drum.

The controller may be configured to block driving the induction module while water is accommodated in the tub after the heating cycle.

### Advantageous Effects

According to the present disclosure, at least one of a wash cycle and a rinse cycle may be performed while a drum is heated with an induction module.

According to the present disclosure, an excessive load may be prevented from being applied to the drive part that spins the drum even when the water is heated by the induction module with the clothing submerged in water.

According to the present disclosure, bubbles may be prevented from being generated or bubbles that have been generated may be removed even when the drum is spun with detergent added to the water.

According to the present disclosure, when a heating cycle is performed, delays in washing may be prevented, energy efficiency may be improved, and water waste may be prevented.

### DESCRIPTION OF DRAWINGS

FIG. 1 illustrates an embodiment of a heating cycle in which a conventional laundry treating apparatus heats clothing with water.

FIG. 2 illustrates an actual operation rate of a drive part when a conventional laundry treating apparatus performs a heating cycle.

FIG. 3 illustrates a control method for a conventional laundry treating apparatus performing a heating cycle.

FIG. 4 illustrates a configuration of a laundry treating apparatus according to the present disclosure.

FIG. 5 illustrates one embodiment of an induction module I of the laundry treating apparatus according to the present disclosure.

FIG. 6 illustrates a base 100 of the induction module.

FIG. 7 illustrates the structure of a coil 600 of the induction module.

FIG. 8 illustrates a specific structure of a magnet cover 320.

FIG. 9 illustrates the arrangement of the coil 600 and permanent magnets 310 provided in the induction module I.

FIG. 10 illustrates the laundry treating apparatus according to the present disclosure performing a heating cycle in which clothing is heated with water.

FIG. 11 illustrates a driving method of a drive part that generates a high load on a drum.

FIG. 12 illustrates a driving method of the drive part that generates lower load on the drum.

FIG. 13 illustrates the condition inside the drum when the actual operation rate of the drive part is increased in the heating cycle of the laundry treating apparatus according to the present disclosure.

FIG. 14 illustrates a control method for the drive part when the temperature is low during operation of the heating cycle.

FIG. 15 illustrates a control method for the drum when the temperature is low during operation of the heating cycle.

FIG. 16 illustrates a control method for the drive part when the temperature is low during operation of the heating cycle.

FIG. 17 illustrates a control method for the drum when the temperature is low during operation of the heating cycle.

FIG. 18 illustrates a control method for the laundry treating apparatus according to the present disclosure.

FIG. 19 illustrates an operation method for a heating wash cycle of the laundry treating apparatus according to the present disclosure.

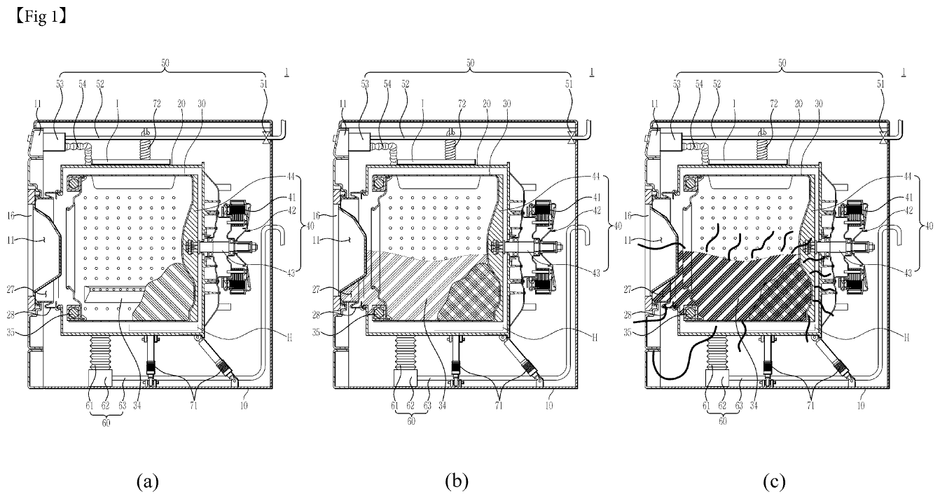

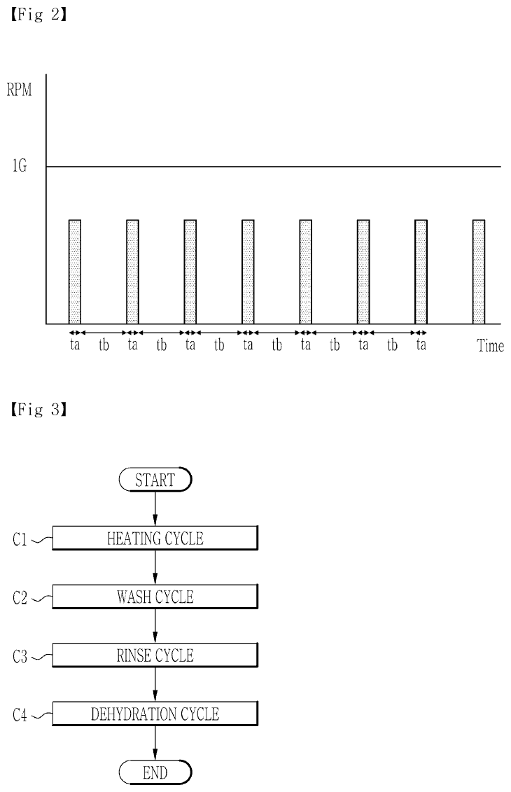

### BEST MODE

Hereinafter, embodiments disclosed herein will be described in detail with reference to the accompanying drawings. In the present disclosure, the same or similar reference numerals are assigned to the same or similar elements in different embodiments, and each of them will be described only once. As used herein, the singular forms include plural forms unless the context clearly indicates otherwise. In addition, in describing the embodiments disclosed herein, when it is determined that the detailed description of the related known technology may obscure the gist of the embodiments, the detailed description thereof will be omitted. In addition, it should be noted that the accompanying drawings are merely intended to provide a further understanding of the embodiments disclosed in the present disclosure and are not to be construed as limiting the technical ideas disclosed herein.

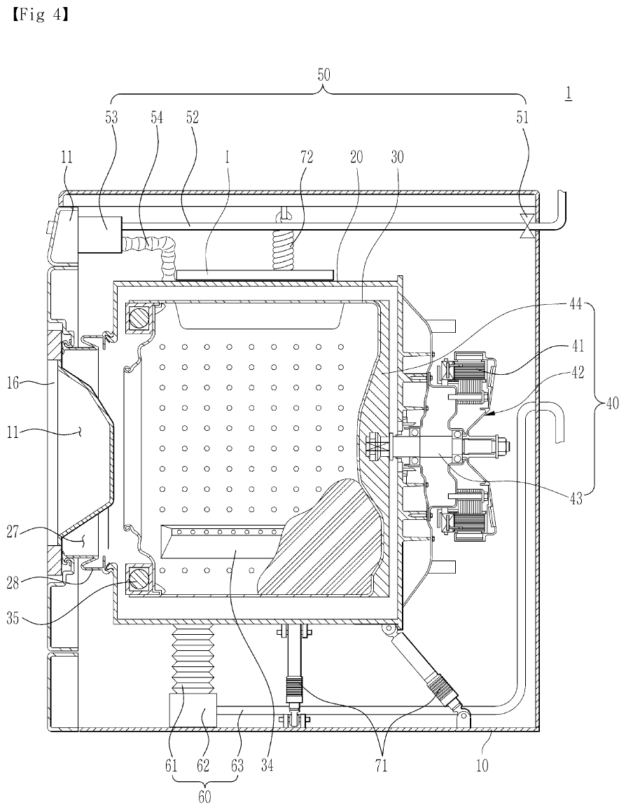

FIG. 4 illustrates a configuration of a laundry treating apparatus according to the present disclosure.

The laundry treating apparatus 1 according to the present disclosure may include a cabinet 10 defining an exterior, a tub 20 arranged inside the cabinet, and a drum 30 rotatably arranged inside the tub 20 to accommodate clothing (or objects to be dried or objects to be refreshed).

The cabinet 10 may include an opening 17 provided at a front of the cabinet 10 to allow the clothing (or objects to be dried or objects to be refreshed) to be put in and retrieved therethrough. The cabinet 10 may include a door 16 rotatably mounted to the cabinet to open and close the opening 17.

The door 16 may include an annular door frame 161 and a transparent window 162 provided in a center portion of the door frame.

The tub 20 is formed in a cylindrical shape with a longitudinal axis kept parallel to or arranged at 0 to 30° with respect to the bottom surface of the cabinet to define a space in which water may be stored. It includes a tub inlet 27 in the front thereof for communication with the opening 17.

The tub 20 may be supported by a support part 70 and fixed inside the cabinet 10.

The support part 70 may include a damper 71 supporting a lower portion of the tub 20, and a spring 72 supporting an upper portion of the tub 20.

Accordingly, vibrations transmitted to the tub 20 by spinning of the drum 30 may be attenuated.

The drum 30 is formed in a cylindrical shape with a longitudinal axis kept parallel to or arranged at 0 to 30° with respect to the bottom surface of the cabinet to accommodate clothing (or objects to be dried or objects to be refreshed), and may include a drum inlet 31 in the front thereof for communication with the tub inlet 27.

Thus, a user may insert clothing (or objects to be dried or objects to be refreshed) into the inner space of the drum 30 or withdraw clothing (or objects to be dried or objects to be refreshed) from the inner space of the drum 30 through the opening, the tub inlet 27 and the drum inlet 31.

The drum 30 may also include a drum outer circumferential surface 32 arranged to accommodate the clothing, and a drum rear surface 33 provided at the rear of the drum and coupled to a drive part 40.

The drum outer circumferential surface 32 includes multiple through holes, which are provided to allow water stored in the tub 20 to flow into the drum, as well as to allow water discharged from the clothing (or objects to be dried or objects to be refreshed) to drain into the inner space of the tub 20.

The inner circumferential surface of the drum 30 may include lifters 34 arranged to agitate the clothing (or objects to be dried or objects to be refreshed) while the drum spins.

The drum 30 may further include a balancer 35 coupled to the front of the outer circumferential surface 32 to compensate for maldistribution inside the drum 30.

Multiple balls or fluids having a mass to compensate for the maldistribution may be accommodated in the balancer 35.

The laundry treating apparatus 1 according to the present disclosure may include a drive part 40 configured to spin the drum 30.

The drive part 40 may be coupled to the tub 20 to spin the drum 30. The drive part 40 may include a stator 41 fixed to the rear surface of the tub 20 to generate a rotating magnetic field, a rotor 42 rotated by electromagnetic interaction with the stator, and a shaft 43 arranged through the rear surface of the tub 20 to connect the drum rear surface 33 and the rotor 42.

Further, the drive part 40 may further include a spider 44 coupled to the drum rear surface 33 to spin the drum 30.

The spider 44 may be provided by extending the shaft 43, and may have one surface coupled to the drum rear surface 33 and coupled to the rotor 42 via the shaft 43.

The laundry treating apparatus 1 according to one embodiment of the present disclosure may further include a water supply part 50 supplied with water from an outside.

The water supply part 50 may include a water supply valve 51 coupled to the cabinet 10 and communicating with an external water source, a water supply pipe 52 extending from the water supply valve 51 to supply water, a detergent box 53 arranged to receive water from the water supply pipe 52 and store detergent, and a supply pipe 54 connecting the detergent box 53 to the tub 20 to supply at least one of the water and the detergent to the tub 20.

The detergent accommodated in the detergent box 53 may be diluted in the water from the water supply pipe 52 and supplied to the tub 20 through the supply pipe 53.

The detergent box 53 may include a housing fixed in the space between the top of the tub 20 and the cabinet 10, and a detergent accommodation portion disposed in the housing to be withdrawn forward.

The laundry treating apparatus according to one embodiment of the present disclosure may further include a drainage part 60 arranged to drain water inside the tub 20 to the outside of the cabinet 10.

The drainage part 60 may include a drainpipe 61 arranged to drain water from the tub 20, a drain pump 62 connected to the drainpipe 61 to provide power to drain the water to the outside of the cabinet 10, and an extension pipe 53 extending from the drain pump 62 to the outside of the cabinet 10.

To facilitate the draining of water from the tub 20 using gravity, the drain pump 62 and the drain pipe 61 may be arranged under the tub 20.

A gasket 28 may be arranged between the opening of the cabinet 10 and the tub inlet 27. The gasket 28 may prevent water inside the tub 20 from leaking into the cabinet 10. Further, the gasket 28 may be formed of an elastic material to prevent vibration of the tub 20 from being transmitted to the cabinet 10.

The laundry treating apparatus 1 according to the present disclosure may include an input panel 11 arranged on the front side to receive instructions for operation of the laundry treating apparatus.

The input panel 11 may be configured to receive a set of instructions for supply of power to the laundry treating apparatus or for execution of a wash cycle, a drying cycle, or the like in which the laundry treating apparatus may wash or dry clothing.

The input panel 11 may be provided as a user interface, and may include a display such as a liquid crystal display or an electric light to display information about the laundry treating apparatus.

The laundry treating apparatus according to the present disclosure may be configured to heat water, dry clothing (or objects to be dried or objects to be refreshed), and refresh (steam) the clothing.

To this end, the laundry treating apparatus 1 according to the present disclosure may include an induction module I configured to heat a drum 30.

The induction module I may be used to perform at least one of washing, drying and refreshing (steaming).

The induction module I may be mounted on the outer circumferential surface of the tub 20, and may include a coil 600 formed by winding wires. The induction module I serves to heat the circumferential surface of the drum 30 through a magnetic field generated by applying an electrical current to the coil 600.

Regarding how the induction module I heats the drum 30, Alternating current whose phase changes is applied to the coil 600 on the outer side of the circumferential surface of the drum 30, and the coil 600 forms a radial alternating magnetic field according to the Ampère's circuital law.

This alternating magnetic field is concentrated toward the drum 30, which is made of a conductor with high permeability. Permeability is the measure of magnetization of a medium in a given magnetic field. Eddy currents are formed in the drum 30 according to Faraday's law of induction. The eddy currents flow through the drum 30 made of the conductor and are converted to joule heat by the resistance of the drum 30. Thereby, the inner wall of the drum 30 is directly heated.

When the inner wall of the drum 30 is directly heated, the temperature of the air inside the drum 30 and the temperature of clothes in contact with the inner wall of the drum 30 increase together. This direct heating of the clothing enables faster drying compared to a drying apparatus that uses only an indirect heating method, such as hot air drying or low-temperature dehumidification drying.

Furthermore, even when the laundry treating apparatus according to the present disclosure is configured as a washing machine as well as a dryer, the water may be heated without having a separate heating wire and flow channel exposed inside the tub 20, and may be continuously in contact with the inner and outer walls of the drum 30. Thus, the water may be heated more quickly than in the case where a separate heater is placed under the tub 20 and used for heating.

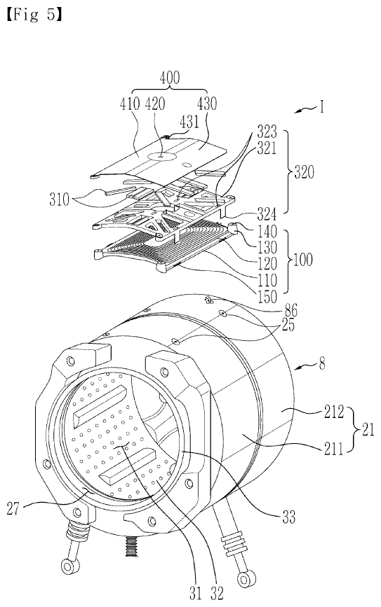

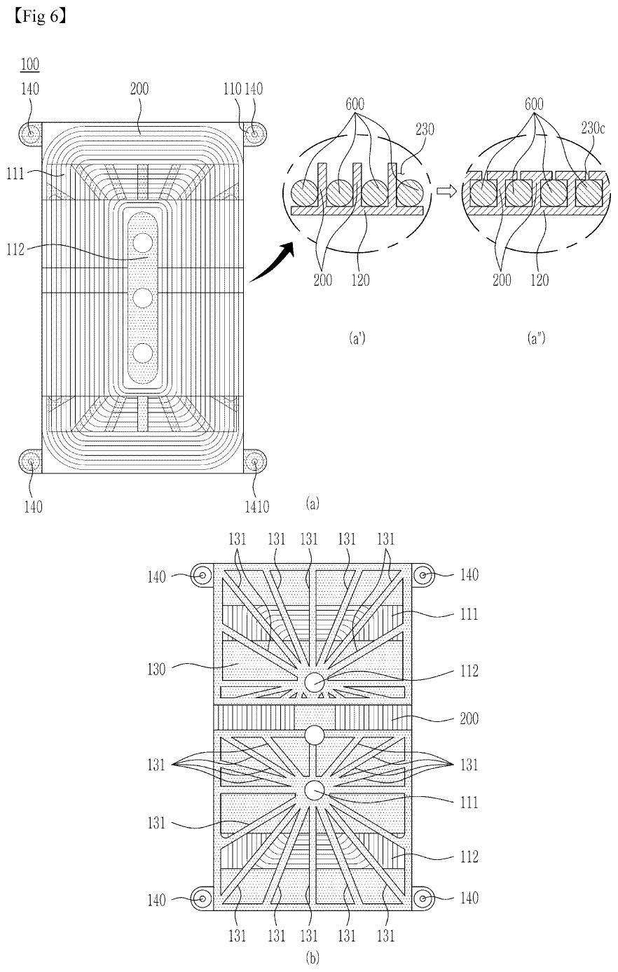

FIG. 5 illustrates one embodiment of an induction module I of the laundry treating apparatus according to the present disclosure.

The induction module I is mounted on the circumferential surface of the tub 20 and heats the circumferential surface of the drum 30 through a magnetic field generated by applying a current to the coil 600 formed by winding wires.

The induction module I may include a base 100 disposed to mount the coil 600 on a top surface of the tub 20. The base 100 may be fixed to the circumferential surface of the tub 20, such that it may be disposed on the upper side of a horizontal surface parallel to the ground by extending through the shaft 43.

The base 100 may be formed in a rectangular plate shape or oblong shape having a predetermined thickness, and may have a front-to-back length longer than a width corresponding to the circumferential direction of the tub 20.

The base 100 may include a base body 110 disposed on the outer circumferential surface of the tub 20, a seating surface 120 arranged on an upper surface of the base body 110, the coil 600 being wound on the seating surface, a bottom surface 130 arranged on a lower surface of the base body 120 to face the outer circumferential surface of the tub 20, and a fixing portion 140 provided to couple the base body 110 to the outer circumferential surface of the tub 20.

At least one of the base body 110 and the seating surface 120 may be curved in cross-section to concentrate the magnetic field generated by the coil 600 on the drum 30.

Further, the base 100 may include a seating rib 200 protruding upward from the seating surface 120 to allow the coil to be wound. The seating rib 200 may extend from an inner side to an outer side of the seating surface 120 in a screw fashion to define an installation space 230 in which the coil 600 is seated or inserted.

In order to concentrate the magnetic field generated by the coil 600 towards the drum 30 and not towards the cabinet 10, the induction module I may include a permanent magnet 310 arranged on the upper side of the base 100 as a bar magnet, and a magnet cover 320 arranged to fix the permanent magnet 310 to the upper portion of the base 100.

The permanent magnet 310 may include permanent magnets spaced apart from each other along the direction in which the coil 600 is wound. The permanent magnet 310 may be positioned on top of the coil 600 and disposed perpendicular to the longitudinal direction of the wires forming the coil 600, such that they are disposed on top of both the inner and outer coils.

The magnet cover 320 may include a housing body 321 having a shape corresponding to the base 100, such as a rectangular plate shape or an oblong shape having a predetermined thickness, a magnet mounting portion 322 provided on the housing body 321 to accommodate the permanent magnet 310, and an air flow hole 323 formed through the housing body 321 and spaced apart from the magnet mounting portion 322.

The magnet mounting portion 322 define a space in which the permanent magnet 310 is accommodated and installed.

The induction module I may include a base cover 400 arranged to fix the magnet cover 320 to the base 100 and to prevent the coil 600 from being displaced.

The base cover 400 may include a cover body 410 having a rectangular plate shape or oblong shape having a predetermined thickness, and an air vent hole 420 provided in a center portion of the cover body 410 to allow heat (air) to flow by convection or to allow a blower fan 500 to be seated therein.

The blower fan 500 may be coupled to the base cover 400 to supply air from the base 100 into the induction module I.

The blower fan 500 may cause air to move through the induction module I to prevent the induction module I from overheating.

Specifically, air may be drawn into the base cover 400 through the air vent hole 420. Inside the induction module, a space is defined between the base cover 400, the magnet coupling part 300, and the base 100, and an air flow space is defined by the air flow hole 323 and the like. Also, the base body 110 is provided with a penetrated portion 111. Thus, air may cool the coil 600 in the inner space and be discharged to the outside of the induction module through the penetrated portion 111.

The magnet cover 320 and the base cover 400 may be provided as separate members, allowing air to flow over the top surface of the permanent magnet 310. Thereby, the permanent magnet 310 may be prevented from overheating.

Further, since the magnet cover 320 and the base cover 400 are separate memebers, the permanent magnet 310 may be easily attached and detached such that the permanent magnet 310 may be easily replaced. Also, since the part fixing the permanent magnet 310 does not have a closed surface, it may be easily injection molded.

Hereinafter, a structure for fixing the base 100, magnet cover 320 and base cover 400 to the tub 20 will be described.

First, the base 100 may include fixing portions 140 arranged at a corner of the base body 110 and provided with a fixing hole 141 into which a screw are inserted. The fixing portions 140 may protrude from both sides of each of the front and rear ends of the base body 110, respectively.

The tub 20 may be provided with multiple coupling portions 25 having a hollow portion communicating with the fixing hole 141.

Furthermore, the magnetic cover 320 may be arranged at a corner of the housing body 321 and may include a magnet fixing portion 330 having a magnetic fixing hole 331 communicating with the fixing hole 141 to allow a screw to be inserted into.

The magnetic fixing portion 330 may protrude from both sides of each of the front and rear ends of the housing body 321, respectively.

Further, the base cover 400 may include cover fixing portions 430 protruding from the front and rear ends of the cover body 410 and provided with a cover fixing hole 431 communicating with the fixing hole 141.

Accordingly, a screw may be arranged through the cover fixing hole 431, magnet fixing hole 331, fixing hole 141 to be fixed to the coupling portion 25.

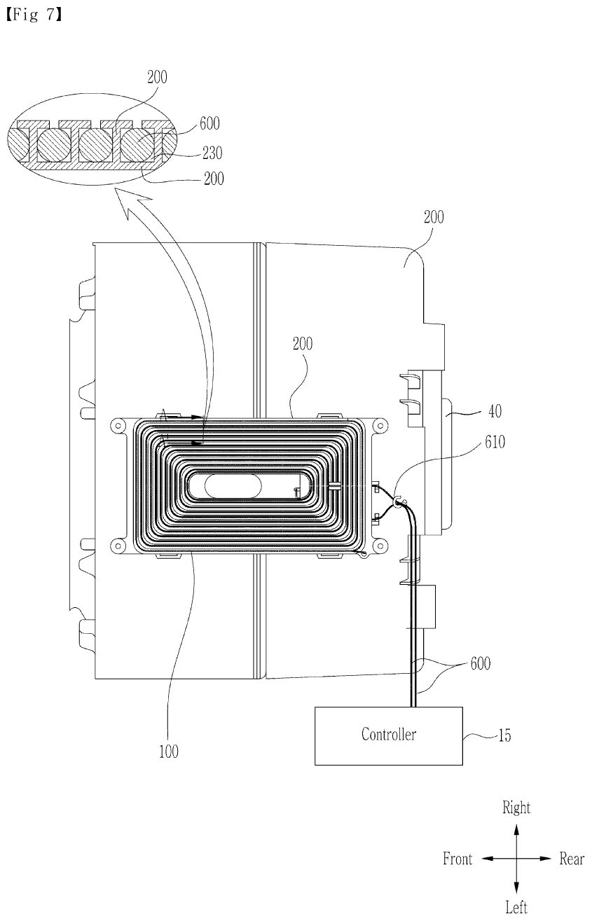

FIG. 6 illustrates a base 100 of the induction module.

The induction module I may include a base 100 serving as a fixing member to fix the coil 600 to the outer circumferential surface of the tub 20. The base may be mounted on the outer circumferential surface of the tub 20 to prevent the coil 600 from being displaced even when the tub 20 vibrates.

FIG. 6-(a) illustrates the top surface of the base 100, while FIG. 6-(b) illustrates the bottom surface of the base 100.

Referring to FIG. 6-(a), the base 100 may include a base body 110 disposed on the outer circumferential surface of the tub 20, a seating surface 120 arranged on an upper surface of the base body 110 to allow the coil 600 to be seated thereon, and a seating rib 200 protruding from the seating surface 120 to allow the coil 600 to be wound to be fixed.

The base body 110 may have a penetrated portion 111 formed in a thickness direction.

The seating rib 200 may extend upward of the penetrated portion 111. In other words, the seating rib 200 may be formed on the seating surface 120 regardless of the shape of the penetrated portion 111.

The seating rib 200 may extend in a direction extending along the perimeter of the seating surface 120 from an inner side to an outer side.

The seating ribs 200 extending from the inner side to the outer side may be spaced apart from each other at a constant distance such that an installation space 230 in which the coil 600 is placed may be defined between the seating ribs 200.

The seating ribs 200 may extend in a circular, elliptical shape with increasing diameters from an inner to an outer side of the seating surface. Alternatively, the seating ribs 200 may extend to form a shape of tracks that increase in area from the inner side to the outer side of the seating surface.

The track shape may be a mixture of straight line portions and curved portions, which may maximize the area of the coil 600 seated on the seating surface 120, compared to a circular shape or an elliptical shape.

The shape of the seating ribs 200 extension on the seating surface 120 may determine the shape of windings of the coil 600.

The seating ribs 200 may protrude or extend upward from the seating surface 120, and may have a height greater than the thickness of the coil 600.

The seating ribs 200 may be spaced apart from each other such that the wound coils 600 do not contact each other, thereby preventing short circuits. Thus, the coils 600 wound on the seating ribs 200 may not need to be coated with a separate insulating film or the thickness of the insulating film may be minimized. Accordingly, production costs may be reduced.

The seating ribs 200 may form a slot having a narrower width than the diameter of the wire of the coil 600 such that the coil 600 is press-fitted. The width of the installation space 230 may be 93% to 97% of the diameter of the wire of the coil 600.

By When press-fitting the coil 600 into the installation space 230, the coil 600 may be held in the installation space 230 even when vibrations of the tub 20 are transmitted to the coil 600. Thus, the coil 600 may be prevented from being displaced from the installation space 230, and noise, which may be generated due to play, may be prevented because movement is suppressed.

The upper ends of the seating ribs 200 may be bent after the coil 600 is inserted to shield at least a portion of the top of the coil 600.

To this end, the upper ends of the seating ribs 200 may be bent or heat-treated.

As such, the upper ends of the seating ribs 200 may form a fixing hook 221 to fix the coil 600.

Referring to FIG. 6-(a), after the coil 600 is press-fitted into the installation space 230, the seating ribs 200 may be melted while the top surface thereof is pressurized. The melted upper ends of the seating ribs 200 may then spread to both sides to cover the top of the coil 600 on both sides.

By fixing the coil 600 in the installation space 230 by press-fitting and melting the upper ends of the seating ribs 200, the paths along which the coil 600 may escape may be physically blocked, the movement of the coil 600 may be prevented. Thereby, noise may be prevented in the event of vibration of the tub 20, and the durability of the parts may be improved by eliminating play between the parts.

While the coil 600 is described as being arranged on the top surface of the base 100, the seating ribs 200 may protrude downward from the base 100 such that the coil 600 is arranged on the bottom surface of the base 100.

FIG. 6-(b) illustrates the bottom surface of the base 100.

The penetrated portion 111 may be exposed on the bottom surface 130 of the base 110.

Also, referring to FIG. 6-(b), the bottom surface of the base 110 may be provided with support bars 131.

The support bars 131 may be arranged to enhance the contact between the base 100 and the outer circumferential surface of the tub 20 and to increase the rigidity of the base 100.

The base 100 may include a through hole 112 in the center of the seating surface 120 on which the coil 600 is not wound. The through hole may include multiple through holes spaced apart from each other at regular intervals in the front-to-back direction of the tub 20.

The support bars 131 may radially extend from the through holes 112 across the base 100.

When the fixing portions 140 are fixed to the coupling portions 25 provided on the outer circumferential surface of the tub 20, the outer circumferential surface of the tub 20 is pressed by the support bars 131. Thus, the base 100 may be coupled to the tub 20 with more pressure than when the entirety of the base 100 is brought into contact with the outer circumferential surface of the tub 20.

As a result, the base 100 does not easily move or disengage from the outer circumferential surface of the tub 20 even when the tub 20 vibrates.

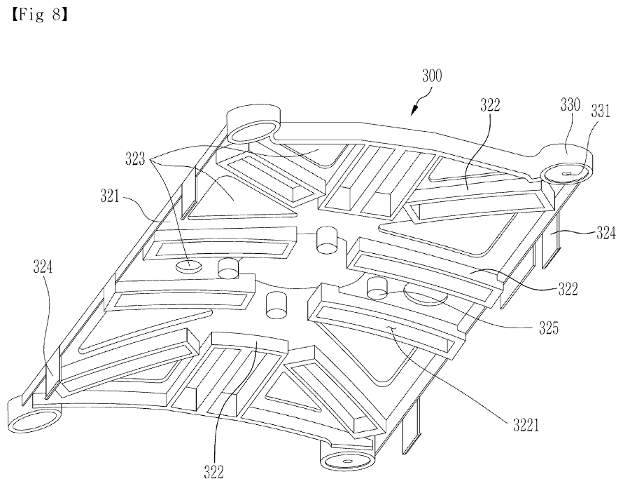

FIG. 7 illustrates the structure of a coil 600 of the induction module.

The seating ribs 200 may extend from positions adjacent the outermost periphery of the seating surface 120 toward the center in a winding manner, and the coil 600 may be arranged between the seating ribs 200 to be wound.

The wire forming the coil 600 may be press-fitted between the seating ribs 200 to make a surface contact.

The laundry treating apparatus according to the present disclosure may include a control panel 15 configured to control the supply of current to the coil 600. Both ends of the coil 600 may be coupled to the control panel 15.

One end of the coil 600 may extend into a through hole provided in the center of the seating surface 120 and the opposite end thereof may extend from the seating surface 120 to an outermost side of the seating ribs 200.

The coil 600 may extend from the control panel 15 to the seating surface 120 through the bottom surface 130 of the base body 110 and be wound on the seating ribs 200. Then, it may be connected to the control panel 15.

In this case, the coil wound on the seating ribs 200 may extend back to the bottom surface 130 and connect to the control panel 15. As the coil 600 is connected to the base 100 through the bottom surface, which is the point at which the outer circumferential surface of the tub 20 is subject to the smallest vibration displacement, the vibration phase difference formed along a wire 81a may be reduced, thereby preventing disconnection and dislodgment.

Both ends of the coil 600 may extend to the rear of the tub 20 to extend to the control panel 15. The rear of the tub 20 is closer to the drive part 40, and may thus have a smaller amplitude.

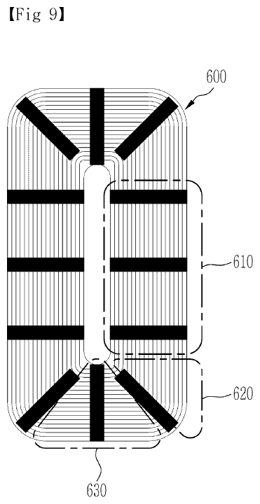

FIG. 8 illustrates a specific structure of the magnet cover 320.

The induction module I may further include a magnet cover 320 coupled to the base 100 to cover the installation space 230.

The magnet cover 320 may include a housing body 321 arranged to be coupled to the top surface of the base 100 and to prevent the coil 600 and permanent magnet 310 from being displaced.

A bottom surface of the housing body 321 may be formed to closely contact the upper ends of the seating ribs 200 of the base 100.

The bottom surface of the magnetic cover 320 may include multiple magnet mounting portions 322 that protrude downward.

The magnet mounting portions 322 may provide space to accommodate the permanent magnet 310, and may closely contact the upper ends of the seating ribs 320 to shield the seating ribs 320 with greater pressure.

Thus, despite the vibration of the tub 20, noise or displacement of the coil 600 resulting from play may be avoided.

Multiple magnet mounting portions 322 may be arranged along the longitudinal direction of the coil 600. Further, they may be arranged perpendicular to the longitudinal direction of the coil 600. Thus, the entire coil may be securely fixed even without pressing the entire coil.

The magnet mounting portions 322 may be integrated with the housing body 321. Thus, the magnet mounting portions 322 press the coil 600 when the magnet cover 320 is coupled to the base 100. Accordingly, there is no need for a separate means or step to press the coil 600.

The permanent magnet 310 may be inserted into the magnet mounting portions 322 to be mounted therein. Thus, once the permanent magnet 310 is fixed to the magnet cover 320, the permanent magnet may be held in place on top of the coil 600 as the magnet cover 320 is coupled to the base housing 100.

Preferably, the permanent magnets 310 may each be disposed at a specific location on the top surface of the coil 600 to efficiently concentrate the magnetic field toward the drum 30. Therefore, if the permanent magnets 310 move with the vibration of the tub 20, it may cause noise as well as a decrease in heating efficiency More specifically, the magnet mounting portion 322 may protrude downward from the bottom surface of the magnet cover 320 to form two side walls facing each other, and may have an open surface 3221 that is open downward such that the bottom surface of the permanent magnet 310 mounted in the magnet mounting portion 322 may face one surface of the coil 600.

In this case, the side-to-side movement of the permanent magnet 310 may be restrained by the side walls, and the open surface 3221 may allow the permanent magnet 310 to be closer to the top surface of the coil 600.

As the permanent magnets 310 are positioned closer to the coil 600, the magnetic field may be more intensively directed toward the drum 30, resulting in stable and uniform heating of the drum 30.

The magnet mounting portion 322 may further include a stop portion protruding inward to prevent the permanent magnet 310 from being displaced downward.

Further, the magnet cover 320 may include detachable hooks 324 arranged at both corners to protrude downward and be detachably coupled to the base 100.

FIG. 9 illustrates the arrangement of the coil 600 and permanent magnets 310 provided in the induction module I.

The coil 600 may be arranged on the outer circumferential surface of the tub 20 in the shape of concentric circles, ellipses, tracks, or the like.

The permanent magnets 310 serve as a blocking member against magnetic field transmission to prevent other surrounding elements besides the drum 30 from being heated, and also serve to concentrate the magnetic field generated by the coil 600 toward the drum 30 to increase heating efficiency.

The permanent magnet 310 may include a bar magnet. Preferably, it may be positioned on top of the coil 600 and disposed perpendicular to the longitudinal direction of the coil 600. This is intended to cover both the inner and outer coils simultaneously.

The permanent magnet 310 may include multiple bar magnets of the same size. The multiple permanent magnets 310 may be spaced apart from each other along the longitudinal direction of the coil 600.

If the permanent magnets 310 are disposed only at certain locations, the amount of magnetic field radiated to the drum 30 will vary from one portion to another of the circumferential surface of the drum 30, making uniform heating difficult. Therefore, the multiple permanent magnets 310 may be spaced apart from each other along the perimeter of the coil 600 in order to direct the magnetic field generated by the coil 600 uniformly toward the drum 30.

Further, given the same number of permanent magnets 310, they are preferably densely disposed on portions of the coil 600 adjacent to the front and rear of the tub 20.

Specifically, the coil 600 may be divided into a first straight portion 610 extending in a front-to-back direction, a curved portion 620 provided at both ends of the straight portion, and a second straight portion 630 disposed at the front and rear.

The first straight portion 610 may be longer than the second straight portion 630, corresponding to the length of the drum 30.

More permanent magnets may be arranged on the curved portion 620 and the second straight portion 630 than on the first straight portion 610. This may allow more magnetic field to be radiated over a smaller area of the coil 600 to uniformly heat the drum 30.

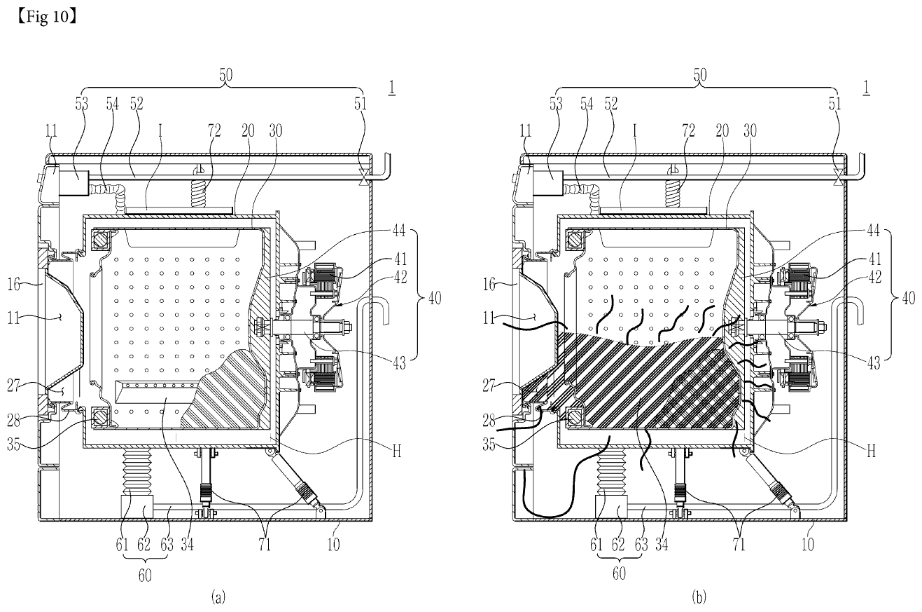

FIG. 10 illustrates the laundry treating apparatus according to the present disclosure performing a heating cycle in which clothing is heated with water.

The laundry treating apparatus according to the present disclosure may further include a water level sensor coupled to the tub 20 to sense a water level in the tub.

The laundry treating apparatus according to the present disclosure may further include a controller P configured to control at least one of the induction module I, the drive part 40, the water supply part 50, and the drainage part 60 to perform any wash course.

The controller P may execute a boil course, an anti-allergy course, or the like, in which a heating cycle is performed to heat the interior of the drum to a sanitization temperature or higher at a water level higher than or equal to a water level at which the clothing is submerged in water.

Referring to FIG. 10-(a), when an instruction for execution of the boil course, or the anti-allergy course, or the like is input, the controller P may perform the heating cycle in which the clothing is heated with water while the clothing is disposed inside the drum 30.

When the heating cycle is performed, the controller P may cause water to be supplied through the water supply part 50 to a heating water level at which the clothing is submerged in water. The heating water level may be set to a water level that is higher than the bottom surface of the drum 30. Alternatively, the heating water level may be set to a water level at which at least a portion of the clothing accommodated in the drum 30 remains submerged.

For example, the heating water level may be set to a water level that ensures that all the clothes accommodated in the drum 30 are submerged.

For example, the heating water level may be set to a water level that is higher than the bottom surface of the drum and is equal to or lower than the height at which the shaft 43 is disposed.

Referring to FIG. 10-(b), the laundry treating apparatus according to the present disclosure may heat the water by driving the induction module I via the controller P.

The controller P may be configured to rotate the drive part 40 together with the induction module I when the induction module I is driven. The controller P may be set to drive the drive part 40 when the induction module I is driven. When the induction module I is driven, the drive part 40 may also rotate to agitate the clothing.

Further, the controller P may be set to interrupt or stop driving the induction module I when driving of the drive part 40 is interrupted or the drum 30 stops.

The controller P may drive the induction module I until the temperature of the water reaches a sanitization temperature or higher.

The controller P may be configured to calculate the temperature of the water accommodated in the tub 20 based on the load generated by the drive part 40.

The laundry treating apparatus according to the present disclosure may further include a temperature sensor coupled to the tub 20 to sense a temperature inside the tub 20 or a temperature of the surface of the drum 30 and communicate the same to the controller P.

The sanitization temperature may be set to a temperature that may improve the cleaning power of the detergent or a temperature at which bacteria or mites that may be present on the clothing begin to be eradicated. The sanitization temperature may correspond to 55 °C or higher.

In the heating cycle, the controller P may heat the inside of the tub 20 to increase the temperature to a target temperature higher than the sanitization temperature.

The target temperature may be set to a temperature at which the clothing may be boiled to kill any bacteria or mites remaining on the clothing. For example, the target temperature may be set to a high temperature near 100 °C, such as 95 °C

When the water accommodated in the tub 20 is heated by the induction module I, heat may be transferred to the drive part 40.

Further, since the drive part 40 is configured to be driven together with the induction module I when the induction module I is driven, the drive part 40 may be further heated by the heat transferred from the tub 20 as well as by the power supplied to spin the drum 30, which result in increase of load.

As a result, when the water accommodated in the tub 20 reaches a target temperature, the drive part 40 may be heated to a higher temperature than the target temperature. Further, when the water accommodated in the tub 20 reaches the sanitization temperature, the drive part 40 may be heated to a higher temperature than the sanitization temperature.

When the drive part 40 is heated above the threshold, the controller P may control the drive part 40 to lower the load to prevent the drive part 40 from being overloaded.

When the drive part 40 reaches a load temperature, the controller P may perform a control operation to reduce the load on the drive part 40. The load temperature may be set to a temperature reached when the water in the tub 20 reaches the sanitization temperature. For example, the load temperature may be set to 70 °C.

The drive part 40 may include a thermistor or the like that senses a temperature related to heat generated by the stator 41, the rotor 42, or the like, and the controller P may sense the temperature of the drive part 40 or the drive part 40 reaching an overloaded state using the thermistor or the like.

In consideration of the amount of load on the drive part 40, the controller P of the laundry treating apparatus according to the present disclosure may drive the drive part 40 to apply more load to the drive part 40 until the water accommodated in the tub 20 reaches the sanitization temperature, and may drive the drive part 40 to apply less load when the water accommodated in the tub 20 reaching the sanitization temperature is heated further.

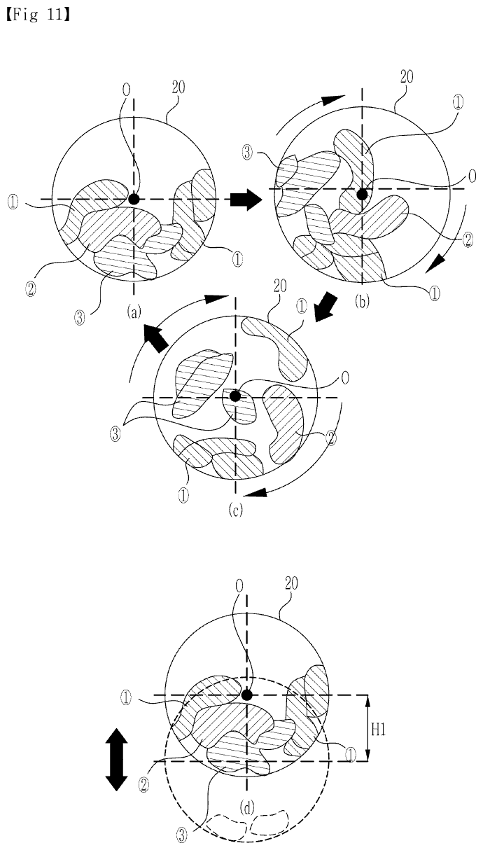

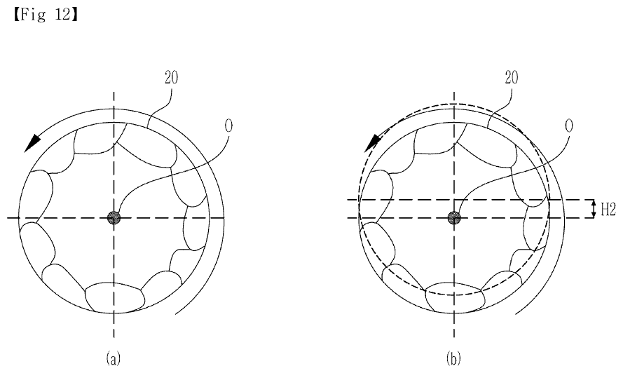

FIG. 11 illustrates a driving method of a drive part that generates a high load on a drum.

In general, when the drum 30 is spun at a higher speed, more load tends to act on the drive part 40.

However, with clothing accommodated in the drum 30 or a portion of the clothing submerged in water, the vibration generated by the drum 30 may exert more load on the drive part 40 than the spin speed of the drum 30.

Referring to FIG. 11-(a), multiple items of clothing may be accommodated in the drum 30.

Referring to FIG. 11-(b), the drum 30 may be spun at a speed slower than a first spin speed (1 RPM) that produces an acceleration force of 1G or more that causes the clothing to rotate in close contact with the drum 30, i.e., a second spin speed (2 RPM). In this case, the clothing may stick to the inner wall of the drum 30 and rise together, and then may fall to the bottom of the drum 30 without reaching the apex of the drum 30.

Referring to FIG. 11-(c), when the drum 30 is continuously spun at the second speed, it may repeatedly cause the clothing to sequentially rise and fall. As a result, an impact may be transmitted to the drum 30, thereby causing the drum 30 to vibrate.

Referring to FIG. 11-(d), when the drum 30 spins at a speed lower than the first speed, the clothing continue to rise above the shaft of the drum 30 and fall to the bottom of the drum 30.

The drum 30 vibrates up and down by a first amplitude H1, which creates additional load on the drive part 40. Further, in the case where the drum 30 is at least partially submerged in water, friction against the water may also be added, and thus the drive part 40 may be subjected to even more load while spinning the drum 30.

On the other hand, when the drum 30 is spun at a speed lower than or equal to the first spin speed, a larger amount of load may act on the drive part 40, but more physical force may be applied to the clothing as to change the position in which the clothing is placed, thereby improving the washing performance.

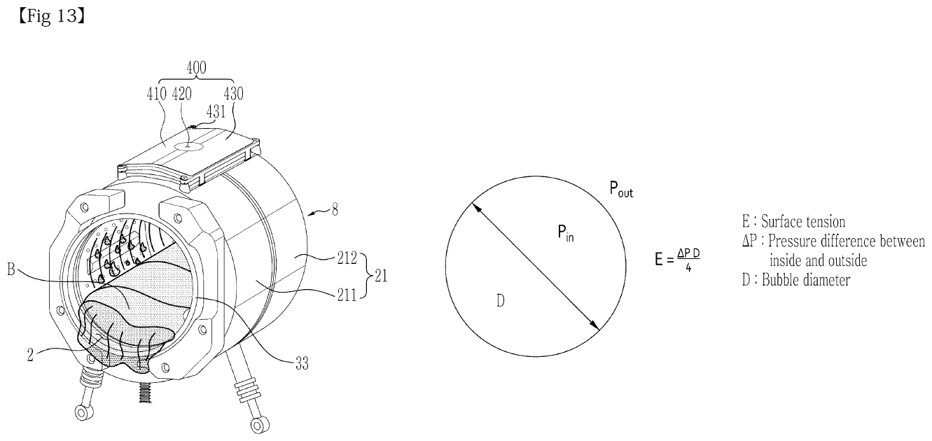

FIG. 12 illustrates a driving method of the drive part that generates lower load on the drum.

Referring to FIG. 12-(a), the drum 30 may be spun at a first spin speed (1 RPM) or higher at which the clothing may be continuously rotated for more than one revolution while being stuck to the inner wall of the drum 30.

When the drum 30 is spun at a speed higher than the first spin speed, the clothing may be continuously rotated while being stuck to the inner wall of the drum 30 by a strong centrifugal force.

As a result, the clothing accommodated in the drum 30 will not detach from the inner wall of the drum 30. Therefore, it will not fall downward from the inner wall of the drum 30, and will not generate additional vibrations in the drum 30.

Referring to FIG. 12-(b), even when the clothing is partially maldistributed in the drum 30, or the drum 30 is partially submerged in water, the clothing will rotate integrally with the drum 30. Accordingly, the drum 30 may vibrate by a second amplitude H2 smaller than the first amplitude.

Therefore, the drive part 40 may be subjected to less load when spinning the drum 30 at the first spin speed or higher than when spinning the drum 30 at a speed lower than the first spin speed.

Further, the laundry treating apparatus according to the present disclosure may prevent the drive part 40 from entering an overloaded state even when the drive part 40 reaches the load temperature or higher, or the temperature of the water inside the tub 20 enters the sanitization temperature or higher and reaches a target temperature, or the drive part 40 is driven at the first spin speed or higher.

Of course, when the drum 30 is spun at the first spin speed or higher, the amount of load applied to the drive part 40 may be reduced, but the washing performance may be greatly reduced because the clothing is stuck to the inner wall of the drum 30 and is neither repositioned nor agitated.

Considering all those cases, the laundry treating apparatus according to the present disclosure may drive the induction module I by varying the RPM of the drive part 40 based on the sanitization temperature or the load temperature.

In the heating cycle, the controller P may arrange at least a portion of the period in which the drum spins at the first spin speed or higher at which the clothing may rotate at least once while being stuck to the inner wall of the drum.

Thereby, the laundry treating apparatus according to the present disclosure may reduce the load on the drive part 40 in the heating cycle to prevent the drive part 40 from entering the overloaded state, and may enable the drive part 40 to rotate longer and more continuously than a conventional laundry treating apparatus.

Rotating the drive part 40 longer and more continuously means driving the induction module I longer and more continuously. Accordingly, the laundry treating apparatus according to the present disclosure may prevent the heating cycle from being delayed even when the heating cycle is carried out using the induction module I.

In the heating cycle, the controller P may repeat the operation of spinning the drum at the first spin speed or higher and then stopping the drum. In other words, after the controller P spins the drum 30 at the first spin speed or higher for a certain period of time, it may stop the drum for a necessary time when it is necessary to reduce the load on the drive part 40 due to an increase in temperature although the drum should be spun at the first spin speed or higher. Then, it may spin the drum 30 at the first spin speed or higher again.

When the drum 30 is spinning or when the induction module I is operating, the water level inside the drum 30 may vary, and the water level sensor may be affected by the magnetic field generated by the induction module I.

Therefore, the controller P may stop the drum 30 as long as necessary in order to sense whether the water level in the tub 20 is in a normal state. Further, even when the drum 30 is driven at the first spin speed, there is room for excessive load to be applied to the drive part 40. Accordingly, the controller P may stop driving the drive part 40 to reduce the temperature of the drive part 40.

In the heating cycle, the controller P may further arrange a period during which the drum is spun at a speed lower than the first spin speed to separate the clothing from the inner wall of the drum. This period is intended to perform at least a portion of the wash cycle even in the heating cycle by washing the clothing in the heating cycle to reduce the execution time of the boil course or the anti-allergy course.

In a temperature range in which large load acts on the drive part 40, the controller P may arrange at least a portion of the period in which it spins the drum 30 at the first spin speed or higher. Thereby, the controller P may reduce the load on the drive part 40, and may thus drive the induction module I longer, thereby increasing the temperature of the water accommodated in the tub 20.

Further, in the temperature range where less load is applied to the drive part 40, additional load can be applied to the drive part 40. Therefore, more periods of spinning the drum 30 at the first spin speed or lower may be arranged. Thereby, the controller P may drive the induction module I to heat the water accommodated in the tub 20 more smoothly, as well as to wash more clothing.

Of course, the controller P may constantly spin the drum 30 at or below the first spin speed in a temperature range where less load is applied. However, since the temperature inside the tub 20 is rising due to driving of the induction module I, at least a portion of the period of spinning the drum 30 at or above the first spin speed may be arranged to prevent temporary excessive load from acting on the drive part 40.

Since the laundry treating apparatus according to the present disclosure drives the induction module I to perform the heating cycle, the drive part 40 is continuously driven regardless of the temperature range. Therefore, the laundry treating apparatus according to the present disclosure may be set such that the actual operation rate of the drive part 40 in the heating cycle increases to 50% or higher. In other words, the laundry treating apparatus according to the present disclosure may be set such that the time during which the drive part 40 is driven is longer than the time during which the drive part 40 is stopped in the heating cycle.

In the heating cycle, the laundry treating apparatus according to the present disclosure may increase the actual operation rate of the drive part 40 and constantly drive the induction module I at a water level at which the clothing is submerged in water.

Further, when the drive part 40 enters the load temperature or approaches a limit temperature at which it may enter the overloaded state, the laundry treating apparatus according to the present disclosure may drive the drive part 40 at the first spin speed or lower to reduce the load on the drive part 40 whenever necessary, thereby preventing the drive part 40 from short-circuiting.

FIG. 13 illustrates the condition inside the drum when the actual operation rate of the drive part is increased in the heating cycle of the laundry treating apparatus according to the present disclosure.

Since the heating cycle is usually performed with water supplied in the first water supply cycle, the condition inside the drum 20 corresponds to the drum containing the detergent introduced.

Unlike the conventional laundry treating apparatus, the laundry treating apparatus according to the present disclosure arranges a period in the heating cycle in which the drive part 40 is driven at an actual operation rate of 50% or higher, while rotating the drive part 40 at a first spin speed or higher.

As a result, bubbles may form inside the drum 30 due to the detergent. As the spin speed of the drive part 40 increases, the amount of bubbles formed increases.

As the amount of bubbles increases inside the drum 30, the bubbles may hinder the spinning of the drum 30, and the drive part 40 may be subjected to more load.

Further, an increase in the amount of the bubbles may cause the bubbles to be discharged through the communication holes (open holes) provided in the tub 20 to leak into the cabinet.

The bubbles will disappear when the internal air pressure or the diameter thereof increases. For example, an increase in the temperature inside the tub 20 may cause the internal temperature or diameter of the bubbles to increase, thereby eliminating the bubbles. Also, when the bubbles come into contact with the surface of the heated drum 30, the bubbles may be removed.

As a result, when the temperature of the water accommodated in the tub 20 or the temperature of the drum 30 rises due to the operation of the induction module I in the heating cycle, generated bubbles may be removed or the generation of bubbles may be blocked even at the high spin speed of the drum 30.

Therefore, based on a removal temperature at which the bubbles are removed, the laundry treating apparatus according to the present disclosure may drive the drum at a low spin speed at a temperature lower than or equal to the removal temperature, and drive the drum at a high spin speed at a temperature higher than the removal temperature.

The removal temperature may be set to correspond to the sanitization temperature.

Also, the removal temperature may be set higher or lower than the sanitization temperature.

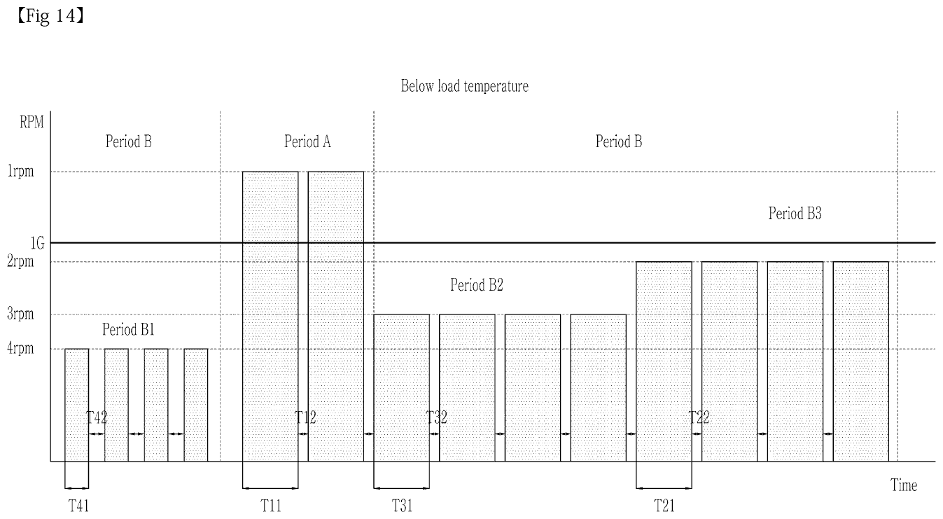

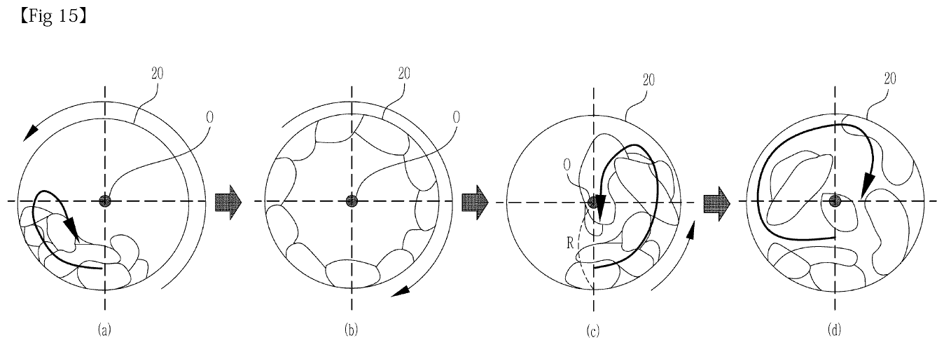

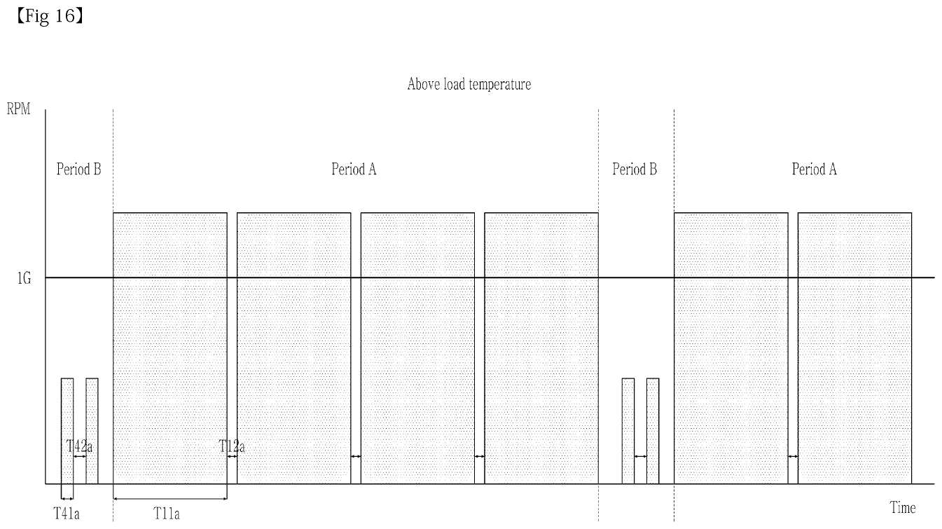

FIG. 14 illustrates a control method for the drive part when the temperature is low during operation of the heating cycle, and FIG. 15 illustrates a control method for the drum when the temperature is low during operation of the heating cycle. The temperature may correspond to any one of the sanitization temperature, the removal temperature, and the load temperature.

The laundry treating apparatus according to the present disclosure may control at least one of the driving speed of the drum 30 and the actual operation rate of the drum 30 differently based on any one of the sanitization temperature, the removal temperature, and the load temperature.

The sanitization temperature and the removal temperature may be defined on the basis of the temperature of water accommodated in the tub 20 or of the drum 30, and the load temperature may be defined on the basis of the temperature of the drive part 40.

The sanitization temperature and the removal temperature may be set to the same temperature, or may be set to different temperatures.

The load temperature may be set to the same temperature as the sanitization temperature and the removal temperature, or may be set to a different temperature. For example, the load temperature may be set higher than the sanitization temperature and the removal temperature.

Hereinafter, a control method will be described in which the laundry treating apparatus according to the present disclosure controls the drive part 40 based on the sanitization temperature.

However, this is merely an embodiment. The laundry treating apparatus according to the present disclosure may be set to distinguishably control the drive part 40 based on the removal temperature rather than the sanitization temperature. Alternatively, the laundry treating apparatus according to the present disclosure may be set to distinguishably control the drive part 40 based on the load temperature, which is a temperature of the drive part 40, rather than a temperature of the inside of the tub 20.

The laundry treating apparatus according to the present disclosure may be set such that the control method of the drive part 40 used when one of the sanitization temperature, the removal temperature, and the load temperature is high is the same as the control method of the drive part 40 used when the temperature is low.

Hereinafter, a description will be given of a case where the control method of the drive part 40 varies depending on whether the temperature of the water accommodated in the tub 20 or the surface temperature of the drum 30 is higher or lower than the sanitization temperature. The same principle may be applied even to a case where the method varies based on the removal temperature or the load temperature.

Referring to FIG. 14, when the temperature inside the tub 20 of the laundry treating apparatus according to the present disclosure is lower than or equal to the sanitization temperature, there is no excessive load exerted on the drive part 40, and thus the drive part 40 is allowed to operate while generating more load.

Therefore, when the temperature inside the tub 20 is lower than or equal to the sanitization temperature, the laundry treating apparatus according to the present disclosure may intensively arrange periods in which the drum 30 spins at a speed lower than the first spin speed, such that the water inside the tub 20 may be heated while the clothing is washed in the heating cycle.

For example, the laundry treating apparatus according to the present disclosure may spin the drum 30 at at least one of a second spin speed lower than the first spin speed, a third spin speed lower than the second spin speed, or a fourth spin speed lower than the third spin speed.

The second spin speed, third spin speed, and fourth spin speed all correspond to a speed at which the clothing cannot be stuck to the inner wall of the drum 30 to continuously rotate. They correspond to a speed at which the clothing is separated from the inner wall of the drum 30.

When the drum spins at one of the second spin speed, third spin speed, or fourth spin speed, the clothing may be washed by physical force applied thereto as the clothing repeatedly sticks to and separates from the inner wall of the drum 30.

Referring to FIG. 15-(a), when the drum 30 is spun at the fourth spin speed, a rolling motion may be performed in the drum 30, in which the clothing repeatedly rotates along the inner wall of the drum 30 and then rolls off without moving up across the shaft or the center O of the drum 30.

When the rolling motion is performed, the surface of the clothing is continuously rubbed, and thus the clothing may be washed by the rubbing.

Referring to FIG. 15-(c), when the drum 30 is spun at the third spin speed, a tumbling motion may be performed in the drum 30, in which the clothing rotates along the inner wall of the drum 30 and then separates from the inner wall of the drum 30 before reaching a high point of the drum 30.

When the tumbling motion is performed, falling energy may be repeatedly applied to the clothing to produce the effect of hitting the clothing 30, such that foreign substances on the clothing can be separated.

Referring to FIG. 15-(d), when the drum 30 is spun at the second spin speed, a shaking motion may be performed the drum 30, in which the clothing rotates along the inner wall of the drum 30 and then separates from the inner wall of the drum 30 in an area past the high point of the drum 30.

When the shaking motion is performed, the arrangement of the clothing inside the drum 30 may be changed such that the surfaces of the clothing evenly contact the inner wall of the drum 30, or that entanglement of the clothing is addressed.

When the temperature inside the tub 20 is lower than the sanitization temperature, the controller P may arrange one or more of a period BI of spinning the drum 30 at the fourth spin speed, a period B2 of spinning the drum at the third spin speed, and a period B4 of spinning the drum at the second spin speed to wash the clothing in various ways.

When the temperature inside the tub 20 is lower than the sanitization temperature, the controller P may arrange a combination of at least two of the period B1 of spinning the drum 30 at the fourth spin speed, the period B2 of spinning the drum at the third spin speed, and the period B4 of spinning the drum at the second spin speed.

Further, when the temperature inside the tub 20 is lower than the sanitization temperature, at least one of the period B1 of spinning the drum 30 at the fourth spin speed, the period B2 of spinning the drum at the third spin speed, or the period B4 of spinning the drum at the second spin speed may be repeated.

Even when any one of the period B1 of spinning the drum 30 at the fourth spin speed, the period B2 of spinning the drum at the third spin speed, and the period B4 of spinning the drum at the second spin speed is arranged, the controller P may set the actual operation rate of the drive part 40 to 50% or higher.

In other words, in the period B1 of spinning the drum 30 at the fourth spin speed, the period B2 of spinning the drum at the third spin speed, and the period B4 of spinning the drum at the second spin speed, the controller P may arrange a period in which the drum 30 rotates at those speed, and a period in which the drum 30 stops.

When the drum 30 spins, the induction module I is driven. When the drum 30 stops, the driving of the induction module I is stopped.

Therefore, when the drum 30 spins at a speed lower than the first spin speed, the controller P may prevent the induction module I and the drive part 40 from overheating by arranging a period in which the drum 30 stops. Further, in the period in which the induction module I stops, the water level of the tub 20 may be sensed normally by the water level sensor to periodically sense the condition inside the tub 20.

Even when the controller P arranges the period in which the drum 30 stops, the period in which the drum 30 spins may be arranged to be longer in every period.

In other words, the controller P may control the drive part 40 such that the time during which the drum 30 spins is longer than the time during which the drum 30 stops in the period B1 of spinning the drum 30 at the fourth spin speed, the period B2 of spinning the drum at the third spin speed, and the period B4 of spinning the drum at the second spin speed.

This is because the drive part 40 is unlikely to overheat when the temperature inside the tub 20 is lower than the sanitization temperature.

The controller P may increase the actual operation rate of the drive part 40 to increase the actual operation rate of the induction module I to heat the water accommodated in the tub 20 more quickly.

In all periods in which the drum 30 spins at a speed lower than the first spin speed, the controller P may set the actual operation rate of the drive part to 50% or higher because bubbles may not be generated inside the tub 20.

Of course, when the temperature inside the tub 20 is lower than the sanitization temperature, the controller P may also arrange a period A in which the controller increases the spin speed of the drum 30 to be higher than or equal to the first spin speed.

Referring to FIG. 15-(b), when the drum 30 is spun at the first spin speed, a filtering motion may be performed, in which the clothing is continuously rotated along the inner wall of the drum 30 inside the drum 30 and is not separated from the inner wall of the drum 30.

When the filtering motion is performed, there is no vibration inside the drum 30 caused by the falling of the clothing, which may reduce the load on the drive part 40.

The controller P may arrange the period A in which the drum 30 spins at the first spin speed or higher, even if the temperature inside the tub 20 is lower than the sanitization temperature, so as to drive the induction module I more intensely to concentrate on heating the water.

Further, the controller P may arrange the period A in which the drum 30 spins at the first spin speed or higher, so as to reduce the load applied to the drive part 40 or the induction module I while performing the wash cycle in the heating cycle.

Further, the controller P may arrange the period A in which the drum 30 spins at the first spin speed or higher, to prevent the amount of electric power used by the laundry treating apparatus from reaching a limit amount of electric power, thereby preventing the heating cycle from being interrupted.

Even when the drum 30 is driven at the first spin speed or higher, a period in which the drum 30 stops may be arranged. Of course, even in this case, the time during which the drum 30 spins may be arranged to be longer than the time during which the drum 30 stops. Thereby, the drive part 40 and the induction module I may be prevented from overheating in the period A of spinning the drum 30 at the first spin speed or higher, and the clothing may be prevented from rotating continuously in an unbalanced state. Further, a period in which the controller P periodically senses the water level of the tub 20 with a water level sensor may be arranged.

Even if the controller P arranges the period A of spinning the drum 30 at the first spin speed or higher when the temperature of the tub 20 is lower than the sanitization temperature, the period A of spinning the drum 30 at the first spin speed or higher may be arranged to be shorter than the period B of spinning the drum 30 at a speed lower than the first spin speed. This is intended to enable intensive washing of the clothing because the drive part 40 or the induction module I is less likely to overheat when the temperature of the tub 20 is lower than the sanitization temperature.

Referring to FIG. 14, in one embodiment in which the above-described control method is applied, the controller P may repeat twice or more an operation of spinning the drum 30 for 13 seconds and stopping the drum 30 for 7 seconds in the period B1 of spinning the drum 30 at the fourth spin speed.

In the period A of spinning the drum 30 at the first spin speed, an operation of spinning the drum 30 for 26 seconds and stopping the drum 30 for 4 seconds may be repeated once or more.

In the period B2 of spinning the drum 30 at the third spin speed, an operation of spinning the drum 30 for 26 seconds and stopping the drum for 4 seconds may be repeated twice or more.

In the period B3 of spinning the drum 30 at the second spin speed, the operation of spinning the drum 30 for 26 seconds and stopping the drum for 4 seconds may be performed once or more.

As these operations are performed, the water inside the tub 20 may be continuously heated, the clothing may be washed, and the load on the drive part 40 may be reduced or maintained.

In the period B1 of spinning the drum 30 at the fourth spin speed, the actual operation rate of the drum may be low in consideration of the fact that the duration of friction of the clothing against the water or the like is greater than in the other periods. However, even in this case, the actual operation rate of the drum may be set to 50% or higher.

Accordingly, when the drive part 40 can handle a greater load, the controller P may sufficiently perform the wash cycle in the heating cycle by washing the clothing, even if additional vibration is generated in the drive part 40.

From another point of view of the above-described control method, when the temperature inside the tub 20 corresponds to a temperature lower than the load temperature, which is lower than the sanitization temperature, in the heating cycle, the controller P may arrange the period A of spinning the drum 30 at the first spin speed or higher to be shorter than the period B of spinning the drum 30 at a speed lower than the first spin speed.

when the temperature inside the tub 20 corresponds to a temperature lower than the load temperature, which is lower than the sanitization temperature, in the heating cycle, the controller P may set the time for continuously spinning the drum at the first spin speed or lower to be longer than or equal to the time for continuously spinning the drum at the first spin speed or higher.

In other words, the total time of the period B in which the drum 30 spins at a speed lower than the first spin speed may be set to be longer than the total time of the period A in which the drum 30 spins at the first spin speed or higher.

Thus, even when the heating cycle is performed, the laundry treating apparatus according to the present disclosure may wash the clothing by arranging the period B of spinning the drum 30 at a speed lower than the first spin speed to be longer in the case where the temperature inside the tub 20 is low, and thus excessive load is not exerted on the drive part 40.

As a result, the laundry treating apparatus according to the present disclosure may perform the wash cycle and the heating cycle simultaneously.

In the case where the temperature inside the tub 20 is lower than the sanitization temperature, and thus excessive load is not exerted on the drive part 40, the laundry treating apparatus according to the present disclosure may increase the proportion of time during which the clothing is rubbed or falling out of the drum 30 while the water is being heated over the time during which the clothing sticks to the inner wall of the drum and rotates.

In the heating cycle, the controller P may reduce the load on the drive part 40 by repeatedly performing the operation of spinning the drum 30 at the first spin speed or higher and stopping the drum, and may wash the clothing by repeatedly performing the operation of spinning the drum 30 at a speed lower than the first spin speed and stopping the drum. The controller P may periodically stop the drum 30 to refresh the drive part 40 and at the same time check the water level in the tub 20.

Further, when stopping and restarting the drum 30, the controller P may change the direction of spinning of the drum 30 to agitate the clothing to prevent only certain parts of the clothing from being heated or over-washed.

In this process, as long as the temperature inside the tub 20 is lower than the sanitization temperature, the number of times the operation of spinning the drum 30 at the first spin speed or higher and stopping the drum is repeated may be set to be less than the number of times the operation of spinning the drum 30 at a speed lower than the first spin speed and stopping the drum is repeated. In other words, when the temperature inside the tub 20 is low, the number of times the drum spins and stops may be set to be greater than the number of times the drum spins at a speed lower than the first spin speed and stops, in order to concentrate more on washing the clothing.

The controller P may spin the drum 30 in various speed periods in spinning the drum 30 at a speed lower than the first spin speed. This is because not only the physical force applied to the clothing but also the duration of the physical force varies depending on the spin speed of the drum 30, and thus the clothing may be washed using various methods.

For example, when the drum 30 is spun at a speed lower than the first spin speed, the controller P may arrange at least a portion of the period B3 of spinning the drum 30 at the second spin speed, which is lower than the first spin speed, and the period B2 of spinning the drum 30 at the third spin speed, which is lower than the second spin speed.

In the period B3 of spinning the drum 30 at the second spin speed, the controller P may stop spinning the drum 30 after spinning the drum 30 at the second spin speed.

In the period B3 of spinning the drum 30 at the second spin speed, the controller P may arrange a period in which spinning of the drum is stopped, which stops the driving of the induction module I. Thereby, the induction module I may be prevented from being overloaded, and the drive part 40 may be prevented from overheating. Also, a change in the water level inside the tub 20 may be sensed using the water level sensor.

In addition, in the period B4 of spinning the drum 30 at the third spin speed, the spinning of the drum 30 may be stopped after the drum 30 is spun at the third spin speed.

In the period B2 of spinning the drum 30 at the third spin speed, the controller P may arrange a period in which spinning of the drum is stopped, which stops the driving of the induction module I. Thereby, the induction module I may be prevented from being overloaded, and the drive part 40 may be prevented from overheating. Also, a change in the water level inside the tub 20 may be sensed using the water level sensor.

When the temperature inside the tub 20 is lower than the sanitization temperature, and thus the controller P spins the drum 30 at a speed lower than the first the spin speed, it may arrange a combination of the period B2 of spinning the drum 30 at the second spin speed and the period B3 of spinning the drum 30 at the third spin speed.

The controller P may alternately arrange the period B3 of spinning at the second spin speed and the period B2 of spinning at the third spin speed according to an algorithm set in the heating cycle, or may arrange the period B3 of spinning at the second spin speed to be longer or shorter than the period B2 of spinning at the third spin speed.

When the temperature inside the tub 20 is lower than the sanitization temperature, and thus the controller P spins the drum 30 at a speed lower than the first spin speed, the controller may further arrange the period B1 of spinning at the fourth spin speed, which is lower than the third spin speed.

The period B1 in which the drum 30 spins at the fourth spin speed may be arranged in combination with the period B3 in which the drum 30 spins at the second spin speed and the period B2 in which the drum 30 spins at the third spin speed.

Further, in the period B1 in which the drum 30 spins at the fourth speed, the drum 30 may repeat spinning at the fourth speed and stopping.

Even when the drum 30 is driven at various spin speeds and stopped because the temperature inside the tub 20 is lower than the sanitization temperature, the controller P may set the time during which the drum 30 spins to be longer than the time during which the drum 30 stops in all periods.

In other words, the controller P may set the actual operation rate of the drive part 40 50% in the heating cycle, and thus set the actual operation rate of the induction module I to 50% or higher, thereby continuously increasing the temperature inside the tub 20.

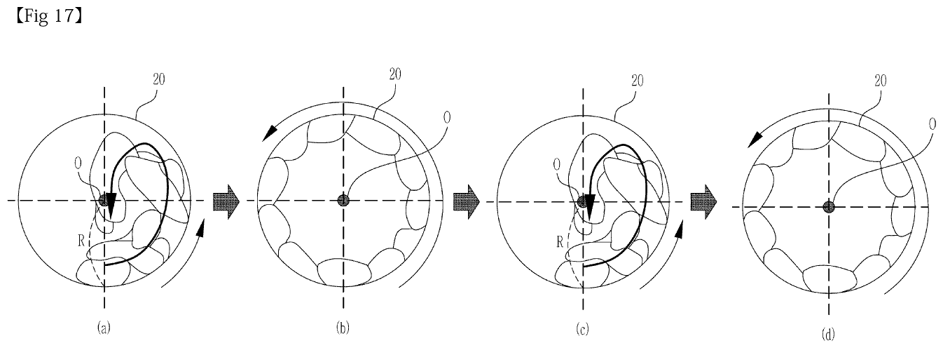

FIG. 16 illustrates a control method for the drive part when the temperature is low during operation of the heating cycle, and FIG. 17 illustrates a control method for the drum when the temperature is low during operation of the heating cycle.

When the temperature of the tub 20 reaches the sanitization temperature or higher, the controller P may set a longer period A in which the drum 30 spins at the first spin speed or higher until the target temperature is reached. Thereby, the drive part 40 may be prevented from being overheated or overloaded even when the drive part 40 is heated by the heat transferred from the tub 20.

The spin time of the drum may be set to be longer and the actual operation rate of the drive part 40 may be set to be higher in the period A of spinning the drum 30 at the first spin speed or higher when the temperature of the tub 20 is higher than the sanitization temperature than in the period A of spinning the drum at the first spin speed or higher when the temperature of the tub 20 is lower than the sanitization temperature.

Since the drive part 40 is being heated by heat transferred from inside the tub 20, the controller P may further minimize the load exerted on the drive part 40 by arranging a longer duration in which the drum 30 is driven at the first spin speed or higher.

The controller P may also set the actual operation rate of the induction module I to a higher rate, such that the temperature inside the tub 20 does not decrease but continuously increases to the target temperature.

When the tub 20 is at the sanitization temperature or higher, the drum 30 may be stopped even in the period A of spinning the drum 30 at the first spin speed or higher. In the period A, the time during which the drum 30 spins may be set to be longer than the time during which the drum 30 stops.

At a temperature higher than or equal to the sanitization temperature, the bubbles cannot be maintained and is removed, and the generation of bubbles may be prevented because the surface of the drum 30 has been sufficiently heated and the temperature inside the tub 20 has also increased. Therefore, at a temperature higher than or equal to the sanitization temperature, the controller P may maintain the drum 30 at the first spin speed or higher for a longer time to intensively increase the temperature inside the tub 20.

The controller P may arrange a period in which the clothing is still washed even when the temperature inside the tub 20 is higher than or equal to the sanitization temperature.

The controller P may arrange the period B of spinning the drum 30 at a speed lower than the first spin speed as well. However, the controller P may keep the spin speed of the drum 30 constant in the period B of spinning the drum 30 at a speed lower than the first spin speed. For example, the drum 30 may only be spun at the fourth spin speed. This is intended to prevent the falling of the clothing inside the drum 30 from causing vibration to prevent an excessive load from being exerted on the drive part 40.

When the temperature inside the tub reaches the sanitization temperature or the drive part 40 reaches a load temperature higher than the sanitization temperature in the heating cycle, the controller P may arrange the period A in which the drum 30 spins at the first spin speed or higher to be longer than the period in which the drum spins at a speed lower than the first spin speed.

When the temperature inside the tub reaches the sanitization temperature or a load temperature lower than the sanitization temperature in the heating cycle, the controller P may set the time for continuously spinning the drum at the first spin speed or higher to be longer than the time for continuously spinning the drum at a speed lower than the first spin speed.

When the temperature inside the tub reaches the sanitization temperature or the temperature of the drive part 40 reaches a temperature higher than or equal to a load temperature higher than the sanitization temperature in the heating cycle, the controller P may arrange a period of spinning the drum at a speed lower than the first spin speed between the periods of spinning the drum at the first spin speed or higher.

When the temperature inside the tub reaches the sanitization temperature or the temperature of the drive part 40 reaches a temperature higher than or equal to the load temperature higher than the sanitization temperature in the heating cycle, the controller P may set the number of times of repeating spinning the drum at the first spin speed or higher and stopping the drum in the heating cycle to be greater than the number of times of repeating spinning the drum at a speed lower than the first spin speed and stopping the drum.

For example, in the period A of spinning the drum 30 at the first spin speed or higher, when the temperature inside the tub 20 corresponds to a temperature higher than or equal to the sanitization temperature, the drum 30 may be set to repeat the operation of spinning for 46 seconds and stopping for 4 seconds twice or more.

The controller P may be set the time during which the drum 30 spins at a speed lower than the first spin speed to be shorter when the temperature inside the tub 20 corresponds to a temperature higher than or equal to the sanitization temperature than when the temperature inside the tub 20 corresponds to a temperature lower than the sanitization temperature.

Further, the controller P may set the time during which the drum 30 stops spinning at a speed lower than the first spin speed to be shorter when the temperature inside the tub 20 corresponds to a temperature higher than or equal to the sanitization temperature than when the temperature inside the tub 20 corresponds to a temperature lower than the sanitization temperature.

Consequently, when the drum 30 spins at a speed lower than the first spin speed, the controller P may set the actual operation rate such that the actual operation rate when the temperature inside the tub 20 corresponding to a temperature higher than or equal to the sanitization temperature is higher than when the temperature inside the tub 20 corresponds to a temperature lower than the sanitization temperature.

Specifically, in the speed period B in which the drum 30 spins at a speed lower than the first spin speed, the drum 30 may be set to repeat the operation of spinning for 11 seconds and stopping for 4 seconds once.

Referring to FIG. 17, when the temperature inside the tub 20 reaches the sanitization temperature or higher, the period B of driving the drum 30 at a speed lower than the first spin speed and the period A of driving the drum 30 at the first spin speed or higher may be repeated. The total time of the period A of driving the drum 30 at the first spin speed or higher may be set to be longer than the total time of the period B of driving the drum 30 at a speed lower than the first spin speed.

Further, when the temperature inside the tub 20 reaches the sanitization temperature or higher, the controller P may set the actual operation rate of the drive part 40 and the induction module I in the period A of driving the drum 30 at the first spin speed or higher to be higher than the actual operation rate of the drive part 40 and the induction module I in the period B of driving the drum 30 at a speed lower than the first spin speed.

Further, when the temperature inside the tub 20 reaches the sanitization temperature or higher, the controller P may set the actual operation rate of the drive part 40 in the period A of driving the drum 30 at the first spin speed or higher to be higher than in any other period.

Thus, even when a larger amount of heat is transferred to the drive part 40 in the heating cycle, the laundry treating apparatus according to the present disclosure may stably drive the induction module I by minimizing the load acting on the drive part 40 when the drum 30 spins.

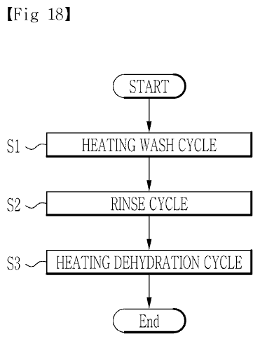

FIG. 18 illustrates a control method for the laundry treating apparatus according to the present disclosure.

When the laundry treating apparatus according to the present disclosure performs a boil course or an anti-allergy course for clothing, it may perform a heating cycle, which includes a period A of spinning the drum 30 at a first spin speed or higher and a period B of spinning the drum 30 at a speed lower than the first spin speed.

Thus, the laundry treating apparatus according to the present disclosure may perform a wash cycle in the heating cycle simultaneously. Therefore, the laundry treating apparatus according to the present disclosure may perform a heating wash cycle S1 when performing the boil course or the anti-allergy course. When the heating wash cycle ends, the laundry treating apparatus according to the present disclosure may perform a rinse cycle S2 in which the drum is re-supplied with water after draining out the water and detergent and then spun and drained. When the rinse cycle S2 ends, the laundry treating apparatus according to the present disclosure may perform a dehydration cycle S3.

In the dehydration cycle S3, the laundry treating apparatus according to the present disclosure may drive the induction module I while driving the drive part 40. Accordingly, the moisture contained in the clothing may be removed more effectively. Therefore, the laundry treating apparatus according to the present disclosure may be regarded as performing a heating dehydration cycle S3.

As a result, the laundry treating apparatus according to the present disclosure may significantly shorten the time to perform the boil course or anti-allergy course by performing the heating wash cycle SI, in which the heating cycle and the wash cycle are performed simultaneously. Further, the laundry treating apparatus according to the present disclosure may shorten the duration of the dehydration cycle by achieving dehydration of the clothing more quickly by raising the temperature inside the drum 30 through the heating dehydration cycle S3.

Of course, the heating cycle may also be performed during the rinse cycle.

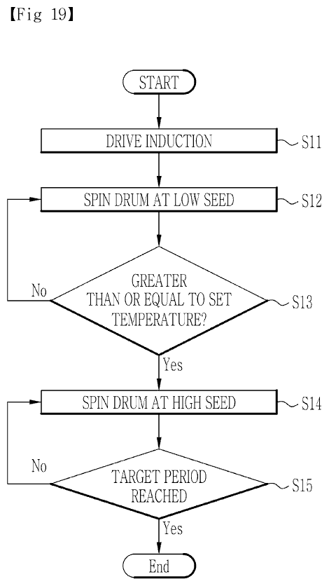

FIG. 19 illustrates an operation method for a heating wash cycle of the laundry treating apparatus according to the present disclosure.

When any course including the heating wash cycle is performed, the laundry treating apparatus according to the present disclosure may spin the drum 30 to sense the amount of laundry, and then perform water supply corresponding to the amount of laundry.

As the water supply is performed, the clothing accommodated in the drum 30 may submerged in water. Thereafter, the laundry treating apparatus according to the present disclosure may perform the heating wash cycle S1.

When performing the heating wash cycle S1, the controller P may perform an induction drive step S11 of driving the induction module I. In the induction drive step S11, the controller P may heat the water accommodated in the tub 20 by heating the drum 30.

The controller P may perform a temperature sensing step S13 of sensing the temperature inside the tub 20 or the temperature of the drive part 40 reaching a set temperature.

The set temperature may correspond to one of the sanitization temperature, removal temperature, and load temperature.

The controller P may perform a low-speed spin step S12 of spinning the drum 30 at a spin speed that generates an acceleration of 1 G or less until the temperature inside the tub 20 or the temperature of the drive part 40 reaches the set temperature.

The low-speed spin step S12 may include some periods in which the drum 30 is spun at a spin speed generating an acceleration of 1 G or more. However, a longer period of time may be set for spinning the drum 30 at a spin speed generating an acceleration of 1 G or less.

In the low-speed spin step S12, the drive part 40 may be set to have an actual operation rate higher than 50% regardless of the spin speed of the drum 30. In other words, the time during which the drum 30 spins may be set to be longer than the time during which the drum 30 stops.

Through the low-speed spin step S12, not only the water in the tub 20 is heated, but also the washing of the clothing may be carried out intensively.

After the temperature inside the tub 20 or the temperature of the drive part 40 reaches the set temperature, the controller P may perform a high-speed spin step S14 of spinning the drum 30 at a spin speed generating an acceleration of 1G or more.

In the high-speed spin step S14, increasing the spin speed of the drum 30 may not cause bubbles to be generated because the temperature inside the tub 20 is already high.

Further, in the high-speed spin step S14, the vibration load exerted on the drive part 40 may be reduced. Accordingly, even when heat is transferred from the tub 20 to the drive part 40, the load or temperature of the drive part 40 may be prevented from reaching a limit.

The controller P may perform a time sensing step S15 of sensing whether the temperature inside the tub 20 has been maintained at a temperature higher than or equal to the sanitization temperature or at the target temperature for a target time.

The controller P may perform the high-speed spin step S14 until the temperature inside the tub 20 is maintained at the temperature higher than or equal to the sanitization temperature or at the target temperature for the target time.

The actual operation rate of the drive part 40 and the induction module I in the high-speed spin step S14 may be set to be higher than that of the drive part 40 and the induction module I in the low-speed spin step S12.

Further, in the high-speed spin step S14, a period of spinning the drum may be arranged such that an acceleration of 1 G or less is generated. Thus, in the high-speed spin step S14, washing of the clothing may still be performed.

However, in the high-speed spin step S14, the duration for which the drum is spun to generate an acceleration of 1 G or less may be set to be shorter than in the low-speed spin step S12.

Further, the total time for which the drum is spun to generate an acceleration of 1 G or less in the high-speed spin step S14 may be set to be shorter than, the total time for which the drum is spun to generate an acceleration of 1 G or less in the low-speed spin step S12.

Further, the actual operation rate of the drive part 40 when the drum is spun to generate an acceleration of 1 G or less in the high-speed spin step S14 may be set to be higher than the actual operation rate of the drive part 40 when the drum is spun to generate an acceleration of 1 G or less in the low-speed spin step S12.

In other words, in the high-speed rotation step S14, the actual operation rate of the drive part 40 and the induction module I may be set to be higher than in the low-speed rotation step S12, and the average spin speed of the drum 30 may be set to be higher and the total time of spinning of the drum 30 may be set to be longer.

Thereby, the load applied to the drive part 40 due to vibration or friction of the drum 30 in the high-speed spin step S14 may be reduced, the drum 30 may be heated more, and the possibility of generating bubbles may be prevented.

When the target period passes, the controller P may terminate the heating wash cycle S1 and perform a post-wash cycle. In other words, since the wash cycle has also been completed in the heating wash cycle S1, the rinse cycle S2 may be performed subsequently.

It will be apparent to those skilled in the art that various modifications and variations can be made in the present disclosure without departing from the spirit and scope of the invention. Thus, it is intended that the present disclosure cover the modifications and variations of the present disclosure provided they come within the scope of the appended claims and their equivalents.

1. A laundry treating apparatus comprising:
a cabinet having an opening in a front thereof;
a tub arranged inside the cabinet to store water;
a water supply valve coupled to the cabinet to supply the water;
a drum rotatably disposed inside the tub to accommodate clothing, the drum comprising a conductor;
a drive part coupled to the tub to spin the drum;
an induction module coupled to an outer circumferential surface of the tub to generate a magnetic field to heat the drum; and
a controller configured to control at least one of the water supply valve, the drive part, or the induction module to perform a heating cycle of heating an inside of the drum to a sanitization temperature or higher at a water level submerging the clothing in water,
wherein, in the heating cycle, the controller is configured to arrange at least a portion of a period of spinning the drum at a first spin speed or higher causing the clothing to be stuck to an inner wall of the drum while rotating one or more revolutions.

2. The laundry treating apparatus of claim 1, wherein, in the heating cycle, the controller is configured to stop driving the induction module when the drum stops.

3. The laundry treating apparatus of claim 1, wherein, in the heating cycle, the controller is configured to repeat stopping the drum after spinning the drum at the first spin speed or higher.

4. The laundry treating apparatus of claim 3, further comprising:
a water level sensor configured to sense a water level in the tub and communicate the same to the controller,
wherein the controller is configured to sense the water level in the tub in a period of stopping the drum.

5. The laundry treating apparatus of claim 1, wherein, in the heating cycle, the controller further is configured to arrange a period of spinning the drum at a speed lower than the first spin speed such that the clothing is separated from the inner wall of the drum.

6. The laundry treating apparatus of claim 5, wherein, based on a temperature inside the tub being higher than or equal to the sanitization temperature in the heating cycle, the controller is configured to arrange the period of spinning the drum at the first spin speed or higher to be longer than the period of spinning the drum at the speed lower than the first spin speed.

7. The laundry treating apparatus of claim 5, wherein, based on a temperature inside the tub being higher than or equal to the sanitization temperature in the heating cycle, the controller is configured to set a time for continuous spinning of the drum at the first spin speed or higher to be longer than a time for continuous spinning of the drum at a speed lower than the first spin speed.

8. The laundry treating apparatus of claim 5, wherein, based on a temperature inside the tub being higher than or equal to the sanitization temperature in the heating cycle, the controller is configured to arrange the period of spinning the drum at the speed lower than the first spin speed in the period of spinning the drum at the first spin speed or higher.

9. The laundry treating apparatus of claim 5, wherein, based on a temperature inside the tub being higher than or equal to the sanitization temperature in the heating cycle, the controller is configured to set the number of times of repeating stopping the drum after spinning the drum at the first spin speed or higher in the heating cycle to be greater than the number of times of repeating stopping the drum after spinning the drum at the speed lower than the first spin speed.

10. The laundry treating apparatus of claim 5, wherein, based on a temperature inside the tub being lower than the sanitization temperature in the heating cycle, the controller is configured to arrange the period of spinning the drum at the first spin speed or higher to be shorter than the period of spinning the drum at the speed lower than the first spin speed.

11. The laundry treating apparatus of claim 5, wherein, based on a temperature inside the tub being lower than the sanitization temperature in the heating cycle, the controller is configured to set a time for continuous spinning of the drum at a speed lower than the first spin speed to be equal to or longer than a time for continuous spinning of the drum at the first spin speed or higher.

12. The laundry treating apparatus of claim 5, wherein, based on a temperature inside the tub being lower than the sanitization temperature in the heating cycle, the controller is configured to set the number of times of repeating stopping the drum after spinning the drum at the first spin speed or higher to be less than the number of times of repeating stopping the drum after spinning the drum at the speed lower than the first spin speed.

13. The laundry treating apparatus of claim 10, wherein, based on the drum being spun at the speed lower than the first spin speed, the controller arranges at least a portion of:
a period of spinning the drum at a second spin speed lower than the first spin speed; and
a period of spinning the drum at a third spin speed lower than the second spin speed.

14. The laundry treating apparatus of claim 13, wherein, in spinning the drum at the speed lower than the first spin speed, the controller arranges at least a portion of:
a period of spinning the drum at the second spin speed and stopping the drum; and
a period of spinning the drum at the third spin speed and stopping the drum.

15. The laundry treating apparatus of claim 13, wherein, in spinning the drum at the speed lower than the first spin speed, the controller arranges a combination of:
the period of spinning the drum at the second spin speed; and
the period of spinning the drum at the third spin speed.

16. The laundry treating apparatus of claim 13, wherein, in spinning the drum at the speed lower than the first spin speed, the controller further is configured to arrange, in combination, a period of spinning the drum at a fourth spin speed lower than the third spin speed.

17. The laundry treating apparatus of claim 6, wherein the controller arranges at least a portion of the period of spinning the drum at the first spin speed or higher causing the clothing to be stuck to the inner wall of the drum while rotating one or more revolutions when the drive part reaches a load temperature, and
wherein the load temperature is configured to be set to a temperature at which bubbles generated in the drum is removed.

18. The laundry treating apparatus of claim 6, wherein the controller arranges at least a portion of the period of spinning the drum at the first spin speed or higher causing the clothing to be stuck to the inner wall of the drum while rotating one or more revolutions when the drive part reaches a load temperature, and
wherein the load temperature is set based on a temperature at which the drive part enters an overloaded state due to heat transferred from the tub during operation of the drive part.

19. A laundry treating apparatus comprising:
a cabinet having an opening in a front thereof;
a tub arranged inside the cabinet to store water;
a water supply valve coupled to the cabinet to supply the water;
a drum rotatably disposed inside the tub to accommodate clothing, the drum comprising a conductor;
a drive part coupled to the tub to spin the drum;
an induction module coupled to an outer circumferential surface of the tub to generate a magnetic field to heat the drum; and
a controller configured to control at least one of the water supply valve, the drive part, or the induction module to perform a heating cycle of heating an inside of the drum to a sanitization temperature or higher at a water level submerging the clothing in water,
wherein, in the heating cycle, the controller is configured to set a time for spinning of the drum with the clothing to be longer than a time for stopping of the drum.

20. The laundry treating apparatus of claim 19, wherein the controller performs a control operation to:
drive the induction module when the drum spins; and
block driving of the induction module when the drum stops.

21. The laundry treating apparatus of claim 19, wherein, based on a temperature inside the tub being higher than or equal to the sanitization temperature in the heating cycle, the controller is configured to set a larger ratio of the time for spinning of the drum to the time for stopping of the drum than based on the temperature inside the tub being lower than the sanitization temperature.

22. The laundry treating apparatus of claim 19, wherein the controller is configured to:
arrange a period of spinning the drum in the heating cycle at a first spin speed or higher and stopping the drum, the first spin speed causing the clothing to be stuck to an inner wall of the drum while rotating at one or more revolutions; and
set a time for spinning of the drum at the first spin speed or higher to be longer than a time for stopping of the drum.

23. The laundry treating apparatus of claim 22, wherein, based on a temperature inside the tub being higher than or equal to the sanitization temperature in the heating cycle, the controller is configured to stop the drum after spinning the drum at the first spin speed more than based on the temperature inside the tub being lower than the sanitization temperature.

24. The laundry treating apparatus of claim 22, wherein the controller is configured to:
further arrange a period of spinning the drum at a speed lower than the first spin speed and stopping the drum in the heating cycle; and
set a time for spinning of the drum at the speed lower than the first spin speed to be longer than a time for stopping of the drum.

25. The laundry treating apparatus of claim 24, wherein, based on a temperature inside the tub being higher than or equal to the sanitization temperature in the heating cycle, the controller is configured to set a smaller ratio of the time for spinning of the drum to the time for stopping of the drum at the speed lower than the first spin speed than based on the temperature inside the tub being lower than the sanitization temperature.

26. The laundry treating apparatus of claim 19, wherein, in a cycle after the heating cycle, the controller is configured to set the time for stopping of the drum to be longer than the time for spinning of the drum.

27. The laundry treating apparatus of claim 26, wherein the controller is configured to block driving the induction module while water is accommodated in the tub after the heating cycle.
